(19) United States
(12) Patent Application Publication
(10) Pub. No.: US 2021/0379095 A1
(43) Pub. Date: Dec. 9, 2021

(54) Methods and Combination Therapy to Treat Biliary Tract Cancer

(71) Applicant: ARRAY BIOPHARMA INC., Boulder, CO (US)

(72) Inventor: Do-Youn Oh, Seoul (KR)

(21) Appl. No.: 16/967,988

(22) PCT Filed: Feb. 12, 2019
(86) PCT No.: PCT/IB19/51131

§ 371 Date: Aug. 6, 2020

Related U.S. Application Data
(60) Provisional application No. 62/728,559, filed on Sep. 7, 2018.
(60) Provisional application No. 62/629,616, filed on Feb. 12, 2018.

(51) Int. Cl. A61K 31/7068; A61K 31/4184; A61K 45/06; A61K 9/00; A61P 35/04

(52) U.S. Cl. CPC A61K 31/7068; A61K 31/4184; A61P 35/04; A61K 9/0053; A61K 45/06

(57) ABSTRACT

This invention relates to a method of treating biliary tract cancer by administering to a patient in need thereof, over a period of time, therapeutic agents comprising a MEK inhibitor or a pharmaceutically acceptable salt thereof, and a fluoropyrimidine-containing therapy, to a patient in need thereof.

### CROSS REFERENCE TO RELATED APPLICATIONS

This application claims the benefit of United States Provisional Application Nos. 62/629,616, filed Feb. 12, 2018 and 62/728,559, filed Sep. 7, 2018, which are incorporated herein by reference in their entirety.

### TECHNICAL FIELD

The present invention relates to methods and combination therapies useful for the treatment of biliary tract cancer. In particular, this invention relates to methods and combination therapies for treating biliary tract cancer by administering a combination therapy consisting essentially of a MEK inhibitor or a pharmaceutically acceptable salt thereof, and a fluoropyrimidine-containing therapy. Pharmaceutical uses of the combination of the present invention are also described.

### BACKGROUND

Approximately 8,000 people in the United States are diagnosed with biliary tract cancer (BTC) each year. BTC is detected at a relatively higher frequency in East and Southeast Asia, where it has been linked to Opisthorchis viverrini and Clonorchis sinensis parasitic infections. Biliary tract cancer affects the epithelial lining of the gallbladder and bile ducts and is often associated with jaundice and pruritus. BTC includes gallbladder cancers and cholangiocarcinomas. Survival rates of patients diagnosed with BTC remain poor, as patients are frequently only diagnosed by the time the cancer has progressed to late stage. Among patients who are diagnosed with early-stage disease BTC, surgical resection is an option for only 10% of patients and the rates of cancer recurrence remain high. For the vast majority of BTC patients, chemotherapy is the first line of treatment. Survival time for patients diagnosed with metastatic BTC or unresectable BTC is predicted to be less than twelve months. BTC has been linked to activating Kirsten Rat Sarcoma (Kras) and epidermal growth factor receptor (EGFR) mutations, as well as loss of function mutations in the Mothers against decapentaplegic homolog 4 (SMAD4), cyclin-dependent kinase 4 inhibitor p16-INK4 (P16INK4A), and tumor protein p53 (P53).

### SUMMARY

The present invention is based on the discovery that a combination of a MEK inhibitor (e.g., binimetinib or a pharmaceutically acceptable salt thereof) and a fluoropyrimidine-containing therapy (e.g., capecitabine) can result in a synergistic therapeutic effect in a subject having biliary tract cancer (e.g., a synergistic reduction in the volume of one or more solid tumors, a synergistic increase in the survival time of the subject, and/or a synergistic increase in the time of stable disease in the subject), e.g., as compared to additive effect of (1) the same dosage of the MEK inhibitor when administered to a subject having biliary tract cancer when administered as a monotherapy, and (2) the same dosage of the fluoropyrimidine-containing therapy when administered to a subject having biliary tract cancer when administered as a monotherapy.

In one embodiment, provided herein is a combination therapy method that comprises administering to a patient in need thereof, over a period of time, therapeutic agents that comprise or consist essentially of or consist of therapeutically effective amounts, independently, of a MEK inhibitor or a pharmaceutically acceptable salt thereof, and a fluoropyrimidine-containing therapy.

In one embodiment, provided herein is a combination therapy method that comprises administering to a patient in need thereof, over a period of time, therapeutic agents that comprise therapeutically effective amounts, independently, of a MEK inhibitor or a pharmaceutically acceptable salt thereof, and a fluoropyrimidine-containing therapy. In one embodiment, the MEK inhibitor is binimetinib or a pharmaceutically acceptable salt thereof. In one embodiment, binimetinib is crystallized binimetinib. In one embodiment, the fluoropyrimidine-containing therapy is capecitabine.

In one embodiment, provided herein is a combination therapy method that comprises administering to a patient in need thereof, over a period of time, therapeutic agents that consist essentially of or consist of therapeutically effective amounts, independently, of a MEK inhibitor or a pharmaceutically acceptable salt thereof, and a fluoropyrimidine-containing therapy. In one embodiment, the MEK inhibitor is binimetinib or a pharmaceutically acceptable salt thereof. In one embodiment, binimetinib is crystallized binimetinib. In one embodiment, the fluoropyrimidine-containing therapy is capecitabine.

In one embodiment, provided herein is a combination therapy method that consists essentially of administering to a patient in need thereof, over a period of time, therapeutic agents that consist essentially of or consist of therapeutically effective amounts, independently, of a MEK inhibitor or a pharmaceutically acceptable salt thereof, and a fluoropyrimidine-containing therapy. In one embodiment, the MEK inhibitor is binimetinib or a pharmaceutically acceptable salt thereof. In one embodiment, binimetinib is crystallized binimetinib. In one embodiment, the fluoropyrimidine-containing therapy is capecitabine.

In one embodiment, provided herein is a combination therapy method that consists essentially of administering to a patient in need thereof, over a period of time, therapeutic agents that consist essentially of or consist of therapeutically effective amounts, independently, of a MEK inhibitor or a pharmaceutically acceptable salt thereof, and a fluoropyrimidine-containing therapy. In one embodiment, the MEK inhibitor is binimetinib or a pharmaceutically acceptable salt thereof. In one embodiment, binimetinib is crystallized binimetinib. In one embodiment, the fluoropyrimidine-containing therapy is capecitabine.

In one embodiment, provided herein is a method for treating biliary tract cancer that comprises administering, over a period of time, an amount of a fluoropyrimidine-containing therapy and an amount of a MEK inhibitor or a pharmaceutically acceptable salt thereof to a patient in need thereof, where the amounts together are effective in treating biliary tract cancer. In one embodiment, the MEK inhibitor is binimetinib. In one embodiment, binimetinib is crystallized binimetinib. In one embodiment, the fluoropyrimidine-containing therapy is capecitabine.

In one embodiment, provided herein is a method for treating biliary tract cancer that consists essentially of administering, over a period of time, therapeutic agents that consist essentially of or consist of an amount of a fluoropyrimidine-containing therapy and an amount of a MEK inhibitor or a pharmaceutically acceptable salt thereof to a patient in need thereof, where the amounts together are effective in treating biliary tract cancer. In one embodiment, the MEK inhibitor is binimetinib. In one embodiment, binimetinib is crystallized binimetinib. In one embodiment, the fluoropyrimidine-containing therapy is capecitabine.

In some embodiments of any of the methods described herein, before the period of time, the patient was treated with one or more therapeutic agents independently selected from chemotherapeutic agents and targeted therapeutic agents. In some embodiments of any of the methods described herein, before the period of time, the patient has been treated with one or more chemotherapeutic agents (e.g., an anti-metabolite (e.g., gemcitabine or capecitabine), a platinum-based chemotherapy (e.g., cisplatin), and optionally, the patient has been previously determined to be non-responsive to treatment with the one or more chemotherapeutic agents. In some embodiments of any of the methods described herein, before the period of time, the patient was treated with a targeted therapeutic agent (e.g., a receptor tyrosine kinase-targeted therapeutic agent (e.g., an EGFR inhibitor), a signal transduction pathway inhibitor (e.g., a MEK inhibitor, a RAS inhibitor, a KRAS inhibitor, a NRAS inhibitor, or a RAF inhibitor), or an angiogenesis-targeted therapy) as a monotherapy, and, optionally, the prior treatment with the targeted therapeutic agent inhibitor as a monotherapy was unsuccessful and/or the patient became resistant to the targeted therapeutic agent. In any of said embodiments, unsuccessful treatments that have been administered to the patient before the period of time can include, but are not limited to, treatments wherein the patient has failed a prior therapy or has been refractory to such prior therapy, and/or wherein the cancer has metastasized or recurred.

In some embodiments of any of the methods described herein, before the period of time, the patient was treated with one or more of a chemotherapy, a targeted anticancer agent, radiation therapy, and surgery, and optionally, the prior treatment was unsuccessful. In some embodiments of any of the methods described herein, before the period of time, the patient was treated with a fluoropyrimidine-containing therapy or a MEK inhibitor as monotherapy. In some embodiments of any of the methods described herein, before the period of time, the patient was treated with a gemcitabine, and optionally, the prior treatment was unsuccessful. In some embodiments of any of the methods described herein, before the period of time, the patient was treated with a MEK inhibitor as a monotherapy, and optionally, the prior treatment was unsuccessful.

In some embodiments of any of the methods described herein, after the period of time, the patient is treated with therapeutic agents that do not consist essentially of a fluoropyrimidine-containing therapy and an amount of a MEK inhibitor which is binimetinib or a pharmaceutically acceptable salt thereof (e.g., therapeutic agents that consist of a fluoropyrimidine-containing therapy and an amount of a MEK inhibitor which is binimetinib or a pharmaceutically acceptable salt thereof)

In some embodiments, administration of the fluoropyrimidine-containing therapy and administration of the MEK inhibitor or a pharmaceutically acceptable salt thereof during the period of time, occurs at substantially the same time. In some embodiments, administration of the fluoropyrimidine-containing therapy to the patient occurs prior to administration of the MEK inhibitor or a pharmaceutically acceptable salt thereof to the patient, during the period of time. In some embodiments, administration of the MEK inhibitor or a pharmaceutically acceptable salt thereof to the patient occurs prior to administration of the fluoropyrimidine-containing therapy to the patient, during the period of time.

In some embodiments, the patient is also administered surgical treatment (e.g., resection of a solid tumor and/or lymph node) during the period of time. In some embodiments, the patient is administered radiation therapy during the period of time. In some embodiments, a patient is administered one or more agents to ameliorate side effects of treatment during the period of time (e.g., one or more of anti-diarrheals (e.g., loperamide), corticosteroids, serotonin antagonists, dopamine antagonists, NK-1 inhibitors, cannabinoids, anti-anxiety drugs (e.g., lorazepam or diazepam), antibiotics, anti-fungal agents, colony-stimulating factor, iron supplements, Procrit, epoetin alfa, darbepoetin alfa, anti-emetics, diuretics, NSAIDs, analgesics, methotrexate, anti-diuretics, probiotics, blood pressure medications, anti-nausea agents, etc.) during the period of time.

In some embodiments of any of the methods described herein, the patient is not administered an additional targeted anticancer agent (e.g., during the period of time. In some embodiments of any of the methods described herein, the subject is not administered a further chemotherapeutic agent during the period of time. In some embodiments of any of the methods described herein, the subject is not administered a non-MEK kinase targeted inhibitor during the period of time. In some embodiments of any of the methods described herein, the patient is not administered one or more of alkylating agents, anthracyclines, cytoskeletal disruptors (e.g., taxanes), epothilones, histone deacetylase inhibitors, topoisomerase I inhibitors, topoisomerase II inhibitors, nucleotide analogs, peptide antibiotics, platinum-based agents, retinoids, and vinca alkaloids and derivatives thereof, during the period of time. In some embodiments of any of the methods described herein, the patient is not administered a c-MET inhibitor during the period of time. In some embodiments of any of the methods described herein, the subject is not administered a CDK4/6 inhibitor during the period of time. In some embodiments of any of the methods described herein, the patient is not administered a PI3K inhibitor during the period of time. In some embodiments of any of the methods described herein, the subject is not administered a BRAF inhibitor (e.g., encorafenib) during the period of time. In some embodiments of any of the methods described herein, the patient is not administered a FGFR inhibitor during the period of time. In some embodiments of any of the methods described herein, the patient is not administered a BCR-ABL inhibitor during the period of time. In some embodiments of any of the methods described herein, the patient is not administered a different anticancer therapy which is a fluoropyrimidine-containing therapy than the one administered during the period of time. In some embodiment of any of the methods described herein, the patient is not administered a different MEK inhibitor than the one administered during the period of time. In some embodiments, the patient is not administered a RAS inhibitor during the period of time. In some embodiments, the patient is not administered a CSR-1R inhibitor during the period of time. In some embodiments, the patient is not administered an EGFR inhibitor during the period of time. In some embodiments, the patient is not administered a RAF inhibitor during the period of time. In some embodiments, the patient is not administered a KRAS inhibitor during the period of time. In some embodiments, the patient is not administered a NRAS inhibitor during the period of time.

In some embodiments, “consisting essentially of,” during the period of time, includes a chemotherapy. In some embodiments, “consisting essentially of,” during the period of time, can include one or more types of chemotherapeutic agents selected from the group of: alkylating agents, anthracyclines, cytoskeletal disruptors (e.g., taxanes), epothilones, histone deacetylase inhibitors, topoisomerase I inhibitors, topoisomerase II inhibitors, nucleotide analogs, additional nucleotide precursor analogs, peptide antibiotics, platinum-based agents, retinoids, vinca alkaloids, and derivatives. In some embodiments, “consisting essentially of” during the period of time, can include treatment with any targeted chemotherapeutic agent. In some embodiments, “consisting essentially of,” during the period of time, can include a surgical treatment and/or chemotherapy. In some embodiments, “consisting essentially of” during the period of time, can include treatment with any targeted chemotherapeutic agent, except for one or more of the following: a EGFR inhibitor, a RAF inhibitor, a RAS inhibitor, a KRAS inhibitor, an NRAS inhibitor, a c-MET inhibitor, a CDK4/6 inhibitor, a PI3K inhibitor, a BRAF inhibitor, a FGFR inhibitor, an additional MEK inhibitor, and a BCR-ABL inhibitor. In some embodiments, “consisting essentially of” during the period of time can include radiation therapy. In one embodiment, the fluoropyrimidine-containing therapy is capecitabine. In some embodiments, the MEK inhibitor is binimetinib. In some embodiments, the MEK inhibitor is crystallized binimetinib.

In another embodiment, the invention provides a method for treating biliary tract cancer comprising or consisting essentially of administering to a patient in need thereof, during a period of time, therapeutic agents that comprise or consist essentially of or consist of an amount of a fluoropyrimidine-containing therapy, and an amount of a MEK inhibitor or a pharmaceutically acceptable salt thereof, wherein the amounts together are effective in treating biliary tract cancer (e.g., during the period of time). In one embodiment, the MEK inhibitor is binimetinib. In one embodiment, binimetinib is crystallized binimetinib. In one embodiment, the fluoropyrimidine-containing therapy is capecitabine.

In another embodiment, the invention provides a method for treating biliary tract cancer comprising or consisting essentially of administering to a patient in need thereof, over a period of time, therapeutic agents that comprise or consist essentially of or consist of an amount of a fluoropyrimidine-containing therapy which is capecitabine, and an amount of a MEK inhibitor which is binimetinib or a pharmaceutically acceptable salt thereof, wherein the amounts together are effective in treating biliary tract cancer (e.g., during the period of time). In one embodiment, binimetinib is crystallized binimetinib. In one embodiment, the fluoropyrimidine-containing therapy is capecitabine.

### BRIEF DESCRIPTION OF THE FIGURES

FIG. 1A is a graph showing the percent cell viability of biliary tract cancer cell lines (SNU245, SNU308, SNU478, SNU869, SNU1079, SNU1196, TFK1, and HuCCT1) after exposure to various concentrations of binimetinib.

FIG. 1B is a bar graph showing the combination index of binimetinib and 5-FU in the biliary tract cancer cell lines SNU245, SNU1196, SNU869, and HuCCT1 (CI<1 at Fa=0.5).

FIG. 1C are western blots showing the expression of thymidylate synthase (TS), programmed death-ligand 1 (PD-L1), and β-actin in cells treated with binimetinib, 5-FU, or a combination of binimetinib and 5-FU.

FIG. 2A is a waterfall plot of tumor shrinkage in patients treated with a combination of binimetinib and capecitabine in the dose escalation part of the Phase 1b study of Example 2, in patients with intrahepatic cholangiocarcinoma (I), extrahepatic cholangiocarcinoma (E), gallbladder cancer (G) and ampulla of Vater cancer (A).

FIG. 2B is a survival curve with at risk table showing the median progression free survival (PFS) and median overall survival (OS) in patients treated with a combination of binimetinib and capecitabine in the dose escalation part of the Phase 1b study of Example 2.

FIG. 2C is a waterfall plot of tumor shrinkage in patients treated with a combination of binimetinib and capecitabine in the dose escalation part of the Phase 1b study of Example 2, in patients with intrahepatic cholangiocarcinoma (I), extrahepatic cholangiocarcinoma (E), gallbladder cancer (G) and ampulla of Vater cancer (A), comparing patients with tumors having mutations in the RAS/RAF/MEK/ERK pathway versus those with wild-type tumors.

FIG. 2D is a survival curve with at risk table showing the median progression free survival (PFS) in patients treated with a combination of binimetinib and capecitabine in the dose escalation part of the Phase 1b study of Example 2, comparing patients with tumors having mutations in the RAS/RAF/MEK/ERK pathway versus those with wild-type tumors.

FIG. 2E is a swimmer plot of treatment duration for patients treated with a combination of binimetinib and capecitabine in the dose escalation part of the Phase 1b study of Example 2, comparing patients with tumors having mutations in the RAS/RAF/MEK/ERK pathway versus those with wild-type tumors.

FIG. 2F is a survival curve with at risk table showing the median overall survival (OS) in patients treated with a combination of binimetinib and capecitabine in the dose escalation part of the Phase 1b study of Example 2, comparing patients with tumors having mutations in the RAS/RAF/MEK/ERK pathway versus those with wild-type tumors.

FIG. 3A is a survival curve with at risk table showing the median progression free survival (PFS) in patients treated with a combination of binimetinib and capecitabine in the dose escalation part of the Phase 1b study of Example 2, comparing patients with higher baseline IL-6 plasma concentrations (>10.3 pg/mL) and lower baseline IL-6 plasma concentrations (≤10.3 pg/mL).

FIG. 3B is a survival curve with at risk table showing the overall free survival (OS) in patients treated with a combination of binimetinib and capecitabine in the dose escalation part of the Phase 1b study of Example 2, comparing patients with higher baseline IL-6 plasma concentrations (>10.3 pg/mL) and lower baseline IL-6 plasma concentrations (≤10.3 pg/mL).

FIG. 3C is a survival curve with at risk table showing the median progression free survival (PFS) in patients treated with two cycles of a combination of binimetinib and capecitabine in the dose escalation part of the Phase 1b study of Example 2, comparing patients with a change in IL-6 plasma concentration of >14.8 pg/mL and ≤14.8 pg/mL between baseline and after the second cycle.

FIG. 3D is a survival curve with at risk table showing the overall free survival (OS) in patients treated with two cycles of a combination of binimetinib and capecitabine in the dose escalation part of the Phase 1b study of Example 2, comparing patients with a change in IL-6 plasma concentration of >14.8 pg/mL and ≤14.8 pg/mL between baseline and after the second cycle.

### DETAILED DESCRIPTION

BTC is detected at a relatively higher frequency in patients in East and Southeast Asia. With only limited treatment options, prognosis remains poor and the overall survival time is only about 8-10 months using the first-line chemotherapy. Chemotherapy and radiation are the mainstay of BTC treatment. For example, combination therapies with 5-fluorouracil, e.g., 5-fluorouracil and leucovorin; 5-fluorouracil and cisplatin; 5-fluorouracil, epirubicin, and cisplatin; 5-fluorouracil an irinotecan; have also been offered to BTC patients (Hezel and Zhu, The Oncologist 13(4): 415-423 (2008).

Gemcitabine, a deoxycytidine analogue, has also been used as a monotherapy for patients with BTC. The combination therapy of gemcitabine and cisplatin is the most commonly used 1st-line chemotherapy. However, a number of subjects having BTC develop resistance to gemcitabine. Without wishing to be bound by theory, the present inventors discovered that the combination therapy of a fluoropyrimidine-containing therapy (e.g., capecitabine) and a MEK inhibitor (e.g., binimetinib) has a synergistic therapeutic effect in BTC cells.

MEK is a key downstream effector of signaling for multiple receptor tyrosine kinases (RTKs) including, e.g., VEGF receptors, CSF1R, and the TAM kinases Mer, AXL, and Tyro3. Combination therapies that include the use of a MEK inhibitor and a fluoropyrimidine-containing therapy were discovered herein to provide for improved anti-tumor responses in a mammal having a cancer (e.g., a gemcitabine-resistant cancer, e.g., a gemcitabine-resistant BTC, that optionally, further has dysregulated MAPK pathway signaling, e.g., as compared to a control tissue). The present invention may be understood more readily by reference to the following detailed description of the preferred embodiments of the invention and the Examples included herein. It is to be understood that the terminology used herein is for the purpose of describing specific embodiments only and is not intended to be limiting. It is further to be understood that unless specifically defined herein, the terminology used herein is to be given its traditional meaning as known in the relevant art.

### General Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

“About” when used to modify a numerically defined parameter (e.g., the dose of a MEK inhibitor or a fluoropyrimidine-containing therapy, or the length of treatment time with a combination therapy described herein) means that the parameter may vary by as much as 10% below or above the stated numerical value for that parameter. For example, a dose of about 5 mg/kg may vary between 4.5 mg/kg and 5.5 mg/kg. “About” when used at the beginning of a listing of parameters is meant to modify each parameter. For example, about 0.5 mg, 0.75 mg or 1.0 mg means about 0.5 mg, about 0.75 mg or about 1.0 mg. Likewise, about 5% or more, 10% or more, 15% or more, 20% or more, and 25% or more means about 5% or more, about 10% or more, about 15% or more, about 20% or more, and about 25% or more.

The term “5-FU” refers to 5-fluorouracil. In one embodiment, 5-FU is formulated for oral administration. In one embodiment, 5-FU is formulated for intravenous administration.

The term “fluoropyrimidine-containing therapy” refers to a therapy selected from (a) 5-FU prodrugs, (b) a combined therapy comprising 5-FU, and (c) a combined therapy comprising a 5-FU prodrug.

The term “5-FU prodrug” refers to a compound that undergoes enzymatic activation by one or more enzyme systems to liberate 5-FU intracellularly. Examples of 5-FU prodrugs include capecitabine (n4-pentyloycarbonyl-5′-deoxy-5-fluorocytidine; Xeloda) which itself is a prodrug of another 5-FU prodrug (doxifluridine), and ftorafur (Tegafur; [R,S-1-1(teterhydrofuran-2-yl)-5-FU]).

The term “DPD inhibitor” refers to a compound that reversibly or irreversibly inhibits dihydropyrimidine dehydrogenase (DPD). Examples of reversible DPD inhibitors include uracil, 5-chloro-2,4-dihydroxypyridine (CDHP; gimeracil), and 3-cyano-2,6-dihydroxypyridine (CNDP). An example of a irreversible inhibitor is 5-ethynyluracil (eniluracil).

The term “combined therapy comprising 5-FU” refers to a combination therapy comprising administration of 5-FU and one or more DPD inhibitors. In one embodiment, the DPD inhibitor is administered prior to administration of 5-FU.

The term “5-FU modulator” refers to an inhibitor of 5-FU phosphoribosylation. An example is oxonic acid, which is a pyrimidine phosphoribosyltransferase inhibitor.

The term “combined therapy comprising 5-FU prodrug” refers to a combination therapy comprising administration of a 5-FU prodrug and one or more a DPD inhibitor and/or a 5-FU modulator. In one embodiment, a “combined therapy comprising 5-FU prodrug” is S-1, which is an oral formulation of ftorafur, oxonic acid and 5-chloro-2,4-dihydroxypyridine (CDHP) at a molar ratio of 1:0.4:1. In one embodiment, a “combined therapy comprising 5-FU prodrug” is the oral formulation BOF A-2, which is a 5-FU prodrug (1-ethoxymethyl 5-FU) combined with the DPD inhibitor 3-cyano-2,6-dihydroxypyridine combined in a 1:1 molar ratio. In one embodiment, a “combined therapy comprising 5-FU prodrug” is the oral formulation UFT, which is a 1:4 molar combination of the 5-FU prodrug ftorafur and the DPD inhibitor uracil.

The terms “cancer”, “cancerous”, or “malignant” refer to or describe the physiological condition in mammals that is typically characterized by unregulated cell growth. Examples of cancer include but are not limited to, pancreatic cancer, breast cancer (e.g., triple-negative breast cancer), mantle cell lymphoma, non-small cell lung cancer, melanoma, colon cancer, esophageal cancer, liposarcoma, multiple myeloma, T-cell leukemia, renal cell carcinoma, gastric cancer, glioblastoma, hepatocellular carcinoma, lung cancer, colorectal cancer, rhabdoid tumor, retinoblastoma proteinpositive cancers, gallbladder cancer, cholangiocarcinoma (e.g., intrahepatic cholangiocarcinoma and extrahepatic cholangiocarcinoma), ampulla of Vater cancer, astrocytomas, glioblastoma multiforme, Bannayan-Zonana syndrome, Cowden disease, Lhermitte-Duclos disease, Wilm's tumor, Ewing's sarcoma, Rhabdomyosarcoma, ependymoma, medulloblastoma, head and neck, kidney cancer, ovarian cancer, prostate cancer, sarcoma, osteosarcoma, giant cell tumor of bone, thyroid, Chronic myelogenous leukemia, Chronic lymphocytic leukemia, Hairy-cell leukemia, acute lymphoblastic leukemia, acute myelogenous leukemia, AML, Chronic neutrophilic leukemia, plasmacytoma, Immunoblastic large cell leukemia, Mantle cell leukemia, Megakaryoblastic leukemia, acute megakaryocytic leukemia, promyelocytic leukemia, Erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, lung cancer, vulval cancer, cervical cancer, endometrial cancer, renal cancer, mesothelioma, salivary gland cancer, nasopharangeal cancer, buccal cancer, cancer of the mouth, GIST (gastrointestinal stromal tumor), NSCLC, or testicular cancer. In some embodiments, the cancer is a T-cell infiltrating cancer. In some embodiments, the cancer is selected from the group consisting of: non-small cell lung cancer, biliary tract cancer, breast cancer, bladder cancer, cervical cancer, malignant mesothelioma, ovarian cancer and pancreatic cancer. In one embodiment, the cancer is biliary tract cancer (BTC).

The term “biliary tract cancer”, also known as “bile duct cancer” or “cholangiocarcinoma”, refers to a cancer that occurs in the bile tract. Biliary tract cancer can form anywhere along the bile ducts and each sub-type of biliary tract cancer is named after the location where the primary cancer begins. Intrahepatic cancers begin inside the bile duct in the liver. Extrahepatic cancers begin from bile ducts outside the liver. Fifty percent of bile duct cancers are Klatskin tumors, which form where the right hepatic duct joins with the left hepatic duct in the liver. Cancers that begin in the common bile duct are called common bile duct cancers. If there are multiple tumors present in different areas of the bile duct this is called multifocal bile duct cancer. The term “biliary tract cancer” also includes gallbladder cancer. The term “biliary tract cancer” also includes ampullary carcinoma. Accordingly, the term “biliary tract cancer” includes extrahepatic cancers, Klatskin tumors, common bile duct cancers, multifocal bile duct cancer, gallbladder cancer and ampullary carcinoma. In one embodiment, the biliary tract cancer is advanced biliary tract cancer. In some embodiments, the biliary tract cancer is selected from the group consisting of: intrahepatic cholangiocarcinoma, extrahepatic cholangiocarcinoma, gallbladder cancer, and ampulla of Vater cancer. In some embodiments, the biliary tract cancer is unresectable. In some embodiments, the biliary tract cancer has reoccurred (e.g., “recurrent disease”). In one embodiment, the biliary tract cancer has a RAS mutation. In one embodiment, the biliary tract cancer has a KRAS mutation. In one embodiment, the biliary tract cancer has a KRAS G12A mutation. In one embodiment, the biliary tract cancer has a KRAS G12C mutation. In one embodiment, the biliary tract cancer has a KRAS G12D mutation. In one embodiment, the biliary tract cancer has a KRAS G12V mutation. In one embodiment, the biliary tract cancer has a NRAS mutation. In one embodiment, the biliary tract cancer has a NRAS Q61L mutation. In one embodiment, the biliary tract cancer has a RAF mutation. In one embodiment, the biliary tract cancer has a BRAF mutation. In one embodiment, the biliary tract cancer has a MAP2K1 mutation. In one embodiment, the biliary tract cancer has a MAP2K1 E203K mutation. In one embodiment, the biliary tract cancer has a MAP2K1 E203V mutation.

The phrases “prior to a period of time” or “before a period of time” refer to (1) the completion of administration of surgery and/or radiation treatment to the subject before the first administration of a therapeutic agent during the period of time, and/or (2) the administration of one or more therapeutic agents to the subject before a first administration of a therapeutic agent in the combination therapy described herein during the period of time, such that the one or more therapeutic agents are present in subtherapeutic and/or undetectable levels in the subject at the time the first administration of a therapeutic agent in the combination therapy is performed during the period of time. In some embodiments, the phrase “prior to a period of time” or “before a period of time” refer to the administration of one or more therapeutic agents to the subject before a first administration of a therapeutic agent in the combination therapy during the period of time, such that the one or more therapeutic agents are present in subtherapeutic levels in the subject at the time the first administration of a therapeutic agent in the combination therapy is performed during the period of time. In some embodiments, the phrase “prior to a period of time” or “before a period of time” refer to the administration of one or more therapeutic agents to the subject before a first administration of a therapeutic agent in the combination therapy during the period of time, such that the one or more therapeutic agents are present in undetectable levels in the subject at the time the first administration of a therapeutic agent in the combination therapy is performed during the period of time. In some embodiments, the phrase “prior to a period of time” or “before a period of time” refer to the administration of one or more therapeutic agents to the subject before a first administration of a therapeutic agent in the combination therapy during the period of time, such that the one or more therapeutic agents are present in subtherapeutic and/or undetectable levels in the subject at the time the first administration of a therapeutic agent in the combination therapy is performed during the period of time.

The phrase “pharmaceutically acceptable” indicates that the substance or composition must be compatible chemically and/or toxicologically, with the other ingredients comprising a formulation, and/or the mammal being treated therewith. Some embodiments relate to the pharmaceutically acceptable salts of the compounds described herein. The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined.

“Administration”, “administering”, “treating”, and “treatment,” as it applies to a patient, individual, animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell.

“Treatment” and “treating”, as used in a clinical setting, is intended for obtaining beneficial or desired clinical results. For purposes of this invention, beneficial or desired clinical results include, but are not limited to, one or more of the following: reducing the proliferation of (or destroying) neoplastic or cancerous cells, inhibiting metastasis of neoplastic cells, shrinking or decreasing the size (e.g., volume) of a tumor, remission of the cancer, decreasing symptoms resulting from the cancer, increasing the quality of life of those suffering from the cancer (e.g., assessed using FACT-G or EORTC-QLQC30), decreasing the dose of other medications required to treat the cancer, delaying the progression of the cancer, and/or prolonging survival of patients having the cancer. For example, treatment can be the diminishment of one or several symptoms of a disorder, such as cancer. Within the meaning of the present invention, the term “treat” also denotes to arrest, delay the onset (i.e., the period prior to clinical manifestation of a disease) and/or reduce the risk of developing or worsening of the cancer. “Treatment” can also mean prolonging survival as compared to expected survival if not receiving treatment, for example, an increase in overall survival (OS) compared to a subject not receiving treatment as described herein, and/or an increase in progression-free survival (PFS) compared to a subject not receiving treatment as described herein. The term “treating” can also mean an improvement in the condition of a subject having the cancer, e.g., one or more of a decrease in the size of one or more tumor(s) in a subject, a decrease or no substantial change in the growth rate of one or more tumor(s) in a subject, a decrease in metastasis in a subject, and an increase in the period of remission for a subject (e.g., as compared to the one or more metric(s) in a subject having a similar cancer receiving no treatment or a different treatment, or as compared to the one or more metric(s) in the same subject prior to treatment). Additional metrics for assessing response to a treatment in a subject having a cancer are disclosed herein below.

The term “subject” includes any organism, preferably an animal, more preferably a mammal (e.g., rat, mouse, dog, cat, and rabbit) and most preferably a human.

A “patient” to be treated according to this invention includes any warm-blooded animal, such as, but not limited to human, monkey or other lower-order primate, horse, dog, rabbit, guinea pig, or mouse. In one embodiment the patient is human. In one embodiment, the patient is a pediatric patient. Those skilled in the medical art are readily able to identify individuals who are afflicted with cancer and who are in need of treatment.

The term “pediatric patient” as used herein refers to a patient under the age of 16 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W. B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994.

The terms “treatment regimen” and “dosing regimen” are used interchangeably to refer to the dose and timing of administration of each therapeutic agent in a combination of the invention.

“Ameliorating” means a lessening or improvement of one or more symptoms as compared to not administering a treatment. “Ameliorating” also includes shortening or reduction in duration of a symptom.

The term “regulatory agency” is a country's agency for the approval of the medical use of pharmaceutical agents with the country. For example, a non-limiting example of a regulatory agency is the U.S. Food and Drug Administration (FDA).

MEK inhibitors in the combination therapies of the invention include, but are not limited to, binimetinib (also called MEK162, ARRY-162, and ARRY 438162), selumetinib (also called AZD6244 and ARRY-142886), trametinib (also called GSK1120212), cobimetinib (also called GDC-0973, XL518, and RG7421), E6201, PD-325901, CI-1040 (also called PD 184352), PD-035901, TAK733, pimasertib (also called AS703026 and MSC1936369B), refametinib (also called RDEA119 and BAY 869766), RO5126766, WX-554, RO4987655 (also called CH4987655), GDC-0973 (also called XL518), AZD8330 (also called ARRY-424704 and ARRY-704), and RO5126766.

In one embodiment, the MEK inhibitor in the combination therapies of the invention is binimetinib or pharmaceutically acceptable salt thereof. Binimetinib has the following structure:

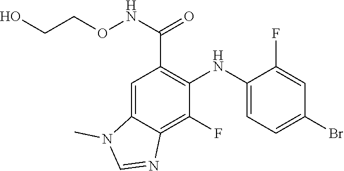 [SMILES: Cn1cnc2c(F)c(Nc3ccc(Br)cc3F)c(C(=O)NOCCO)cc21]

Binimetinib is also known as ARRY-162, ARRY-438162, MEK162, 6-(4-bromo-2-fluorophenylamino)-7-fluoro-3-methyl-3H-benzoimidazole-5-carboxylic acid (2-hydroxyethoxy)-amide, and 5-((4-bromo-2-fluorophenyl)amino)-4-fluoro-N-(2-hydroxyethoxy)-1-methyl-1H-benzimidazole-6-carboxamide. Methods of preparing binimetinib and its pharmaceutically acceptable salts are described in PCT publication No. WO 03/077914, in Example 18 (compound 29111), the disclosure of which is herein incorporated by reference in its entirety. In one embodiment, the MEK inhibitor is binimetinib as the free base. In one embodiment, the MEK inhibitor is a pharmaceutically acceptable salt of binimetinib. In one embodiment, the MEK inhibitor is crystallized binimetinib. Crystallized binimetinib and methods of preparing crystallized binimetinib are described in PCT publication No. WO 2014/063024, the disclosure of which is herein incorporated by reference in its entirety.

In one embodiment, the fluoropyrimidine-containing therapy is a 5-FU prodrug. In one embodiment, the fluoropyrimidine-containing therapy is an oral formulation of a 5-FU prodrug. In one embodiment, the fluoropyrimidine-containing therapy is capecitabine.

The term “combination therapy” as used herein refers to a dosing regimen of two different therapeutically active agents (i.e., the components or combination partners of the combination, i.e., a MEK inhibitor or a pharmaceutically acceptable salt thereof and a fluoropyrimidine-containing therapy) during a period of time, wherein the therapeutically active agents are administered together or separately in a manner prescribed by a medical care taker or according to a regulatory agency as defined herein. In one embodiment, a combination therapy comprises a combination of a MEK inhibitor a pharmaceutically acceptable salt thereof, and a fluoropyrimidine-containing therapy. In one embodiment, a combination therapy consists essentially of a combination of a MEK inhibitor or a pharmaceutically acceptable salt thereof, and a fluoropyrimidine-containing therapy. In one embodiment, a combination therapy comprises a combination of a MEK inhibitor which is binimetinib or a pharmaceutically acceptable salt thereof, and a fluoropyrimidine-containing therapy which is a prodrug of 5-FU which is capecitabine. In one embodiment, a combination therapy consists essentially of a combination of a MEK inhibitor which is binimetinib or a pharmaceutically acceptable salt thereof, and a fluoropyrimidine-containing therapy which is a prodrug of 5-FU which is capecitabine.

As can be appreciated in the art, a combination therapy can be administered to a patient for a period of time. In some embodiments, the period of time occurs following the administration of a different cancer therapeutic treatment/agent or a different combination of cancer therapeutic treatments/agents to the patient. In some embodiments, the period of time occurs before the administration of a different cancer therapeutic treatment/agent or a different combination of cancer therapeutic treatments/agents to the patient. In some embodiments, administration of the fluoropyrimidine-containing therapy and administration of the MEK inhibitor or a pharmaceutically acceptable salt thereof occurs at substantially the same time. In some embodiments, administration of the fluoropyrimidine-containing therapy to the patient occurs prior to administration of binimetinib or a pharmaceutically acceptable salt thereof to the patient, during the period of time. In some embodiments, administration of the MEK inhibitor or a pharmaceutically acceptable salt thereof to the patient occurs prior to administration of the fluoropyrimidine-containing therapy to the patient, during the period of time. In some embodiments, the patient is administered a surgical treatment (e.g., tumor resection and/or lymph node resection) and/or anticancer therapy during the period of time.

A suitable period of time can be determined by one skilled in the art (e.g., a physician). As can be appreciated in the art, a suitable period of time can be determined by one skilled in the art based on one or more of: the stage of disease in the patient, the mass and sex of the patient, clinical trial guidelines (e.g., those on the fda.gov website), and information on the approved drug label. For example a suitable period of time can be, e.g., from 1 week to 2 years, 1 week to 22 months, 1 week to 20 months, 1 week to 18 months, 1 week to 16 months, 1 week to 14 months, 1 week to 12 months, 1 week to 10 months, 1 week to 8 months, 1 week to 6 months, 1 week to 4 months 1 week to 2 months, 1 week to 1 month, 2 weeks to 2 years, 2 weeks to 22 months, 2 weeks to 20 months, 2 weeks to 18 months, 2 weeks to 16 months, 2 weeks to 14 months, 2 weeks to 12 months, 2 weeks to 10 months, 2 weeks to 8 months, 2 weeks to 6 months, 2 weeks to 4 months, 2 weeks to 2 months, 2 weeks to 1 month, 1 month to 2 years, 1 month to 22 months, 1 month to 20 months, 1 month to 18 months, 1 month to 16 months, 1 month to 14 months, 1 month to 12 months, 1 month to 10 months, 1 month to 8 months, 1 month to 6 months, 1 month to 4 months, 1 month to 2 months, 2 months to 2 years, 2 months to 22 months, 2 months to 20 months, 2 months to 18 months, 2 months to 16 months, 2 months to 14 months, 2 months to 12 months, 2 months to 10 months, 2 months to 8 months, 2 months to 6 months, 2 months to 4 months, 3 months to 2 years, 3 months to 22 months, 3 months to 20 months, 3 months to 18 months, 3 months to 16 months, 3 months to 14 months, 3 months to 12 months, 3 months to 10 months, 3 months to 8 months, 3 months to 6 months, 4 months to 2 years, 4 months to 22 months, 4 months to 20 months, 4 months to 18 months, 4 months to 16 months, 4 months to 14 months, 4 months to 12 months, 4 months to 10 months, 4 months to 8 months, 4 months to 6 months, 6 months to 2 years, 6 months to 22 months, 6 months to 20 months, 6 months to 18 months, 6 months to 16 months, 6 months to 14 months, 6 months to 12 months, 6 months to 10 months, 6 months to 8 months, 8 months to 2 years, 8 months to 22 months, 8 months to 20 months, 8 months to 18 months, 8 months to 16 months, 8 months to 14 months, 8 months to 12 months, 8 months to 10 months, 10 months to 2 years, 10 months to 22 months, 10 months to 20 months, 10 months to 18 months, 10 months to 16 months, 10 months to 14 months, 10 months to 12 months, 12 months to 2 years, 12 months to 22 months, 12 months to 20 months, 12 months to 18 months, 12 months to 16 months, or 12 months to 14 months, inclusive.

As used herein, an “effective dosage” or “effective amount” or “therapeutically effective amount” of a drug, compound, or pharmaceutical composition is an amount sufficient to effect any one or more beneficial or desired results. For prophylactic use, beneficial or desired results include reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing incidence or amelioration of one or more symptoms of various diseases or conditions (such as for example cancer), decreasing the dose of other medications required to treat the disease, enhancing the effect of another medication, and/or delaying the progression of the disease. An effective dosage can be administered in one or more administrations. For purposes of this invention, an effective dosage of a drug, compound, or pharmaceutical composition is an amount sufficient to accomplish prophylactic or therapeutic treatment either directly or indirectly. As is understood in the clinical context, an effective dosage of a drug, compound, or pharmaceutical composition may be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved. In reference to the treatment of cancer, an effective amount may also refer to that amount which has the effect of (1) reducing the size of the tumor, (2) inhibiting (that is, slowing to some extent, preferably stopping) tumor metastasis emergence, (3) inhibiting to some extent (that is, slowing to some extent, preferably stopping) tumor growth or tumor invasiveness, and/or (4) relieving to some extent (or, preferably, eliminating) one or more signs or symptoms associated with the cancer. Therapeutic or pharmacological effectiveness of the doses and administration regimens may also be characterized as the ability to induce, enhance, maintain or prolong disease control and/or overall survival in patients with these specific tumors, which may be measured as prolongation of the time before disease progression

The term “BID” as used herein means twice a day.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas.

The term “advanced”, as used herein, as it relates to solid tumors, includes locally advanced (non-metastatic) disease and metastatic disease. Locally advanced solid tumors, which may or may not be treated with curative intent, and metastatic disease, which cannot be treated with curative intent are included within the scope of “advanced solid tumors, as used in the present invention. Those skilled in the art will be able to recognize and diagnose advanced solid tumors in a patient.

“Tumor burden” also referred to as “tumor load”, refers to the total amount of tumor material distributed throughout the body. Tumor burden refers to the total number of cancer cells or the total size of tumor(s), throughout the body, including lymph nodes and bone narrow. Tumor burden can be determined by a variety of methods known in the art, such as, e.g. by measuring the dimensions of tumor(s) upon removal from the subject, e.g., using calipers, or while in the body using imaging techniques, e.g., ultrasound, bone scan, computed tomography (CT) or magnetic resonance imaging (MM) scans.

The term “tumor size” refers to the total size of the tumor (e.g., a solid tumor) which can be measured as the length and width of a tumor, or the volume of the tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT, or Mill scans.

“Individual response” or “response” can be assessed using any endpoint indicating a benefit to the individual, including, without limitation, (1) inhibition, to some extent, of disease progression (e.g., cancer progression), including slowing down or complete arrest; (2) a reduction in tumor size; (3) inhibition (i.e., reduction, slowing down, or complete stopping) of cancer cell infiltration into adjacent peripheral organs and/or tissues; (4) inhibition (i.e. reduction, slowing down, or complete stopping) of metastasis; (5) relief, to some extent, of one or more symptoms associated with the disease or disorder (e.g., cancer); (6) increase or extension in the time of survival, including overall survival and progression-free survival; and/or (7) decreased mortality at a given point of time following treatment.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and/or progression-free survival); resulting in an objective response (including a complete response or a partial response); or improving signs or symptoms of cancer.

An “objective response” or “OR” refers to a measurable response, including complete response (CR) or partial response (PR). An “objective response rate” (ORR) refers to the proportion of patients with tumor size reduction of a predefined amount and for a minimum time period. Generally, ORR refers to the sum of complete response (CR) rate and partial response (PR) rate.

“Complete response” or “CR” as used herein means the disappearance of all signs of cancer (e.g., disappearance of all target lesions) in response to treatment. This does not always mean the cancer has been cured.

As used herein, “partial response” or “PR” refers to a decrease in the size of one or more tumors or lesions, or in the extent of cancer in the body, in response to treatment. For example, in some embodiments, PR refers to at least a 30% decrease in the sum of the longest diameters (SLD) of target lesions, taking as reference the baseline SLD.

“Sustained response” refers to the sustained effect on reducing tumor growth after cessation of a treatment. For example, the tumor size may be the same size or smaller as compared to the size at the beginning of the medicament administration phase. In some embodiments, the sustained response has a duration of at least the same as the treatment duration, at least 1.5×, 2×, 2.5×, or 3× length of the treatment duration, or longer.

As used herein, “progression-free survival” (PFS) refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall survival” (OS) refers to the percentage of individuals in a group who are likely to be alive after a particular duration of time.

“Duration of Response” for purposes of the present invention means the time from documentation of tumor model growth inhibition due to drug treatment to the time of acquisition of a restored growth rate similar to pretreatment growth rate.

By “extending survival” is meant increasing overall or progression-free survival in a treated patient relative to an untreated patient (i.e. relative to a patient not treated with the medicament).

As used herein, “drug related toxicity”, “infusion related reactions” and “immune related adverse events” (“irAE”), and the severity or grades thereof are as exemplified and defined in the National Cancer Institute's Common Terminology Criteria for Adverse Events v 4.0 (NCI CTCAE v 4.0).

“Loss of heterozygosity score” or “LOH score” as used here in, refers to the percentage of genomic LOH in the tumor tissues of an individual. Percentage genomic LOH, and the calculation thereof are described in Swisher et al (The Lancet Oncology, 18(1):75-87, January 2017), the disclosure of which is incorporated herein by reference in its entirety. Exemplary genetic analysis includes, without limitation, DNA sequencing, and Foundation Medicine's NGS-based T5 assay.

“Homologous recombination deficiency score” or “HRD score” as used here in, refers to the unweighted numeric sum of loss of heterozygosity (“LOH”), telomeric allelic imbalance (“TAI”) and large-scale state transitions (“LST”) in the tumor tissues of an individual. HRD score, together with LOH, and LOH score, and the calculation thereof are described in Timms et al, Breast Cancer Res 2014 Dec. 5; 16(6):475, Telli et al Clin Cancer Res; 22(15); 3764-73.2016, the disclosures of which are incorporated herein by reference in their entireties. Exemplary genetic analysis includes, without limitation, DNA sequencing, Myriad's HRD or HRD Plus assay (Mirza et al N Engl J Med 2016 Dec. 1; 375(22):2154-2164, 2016).

The term “tumor proportion score” or “TPS” as used herein refers to the percentage of viable tumor cells showing partial or complete membrane staining in an immunohistochemistry test of a sample. Exemplary samples include, without limitation, a biological sample, a tissue sample, a formalin-fixed paraffin-embedded (FFPE) human tissue sample and a formalin-fixed paraffin-embedded (FFPE) human tumor tissue sample.

In some embodiments, the anti-cancer effects of the methods described herein, including, but not limited to “objective response”, “complete response”, “partial response”, “progressive disease”, “stable disease”, “progression free survival”, “duration of response”, are as defined and assessed by the investigators using RECIST v1.1 (Eisenhauer et al, Eur J of Cancer 2009; 45(2):228-47) in patients with locally advanced or metastatic solid tumors. The disclosures of Eisenhauer et al, Eur J of Cancer 2009; 45(2):228-47 and Scher et al, J Clin Oncol 2016 Apr. 20; 34(12):1402-18 are herein incorporated by references in their entireties.

In some embodiments, the anti-cancer effect of the methods described herein, including, but not limited to “immune-related objective response” (irOR), “immune-related complete response” (irCR), “immune-related partial response” (irCR), “immune-related progressive disease” (irPD), “immune-related stable disease” (irSD), “immune-related progression free survival” (irPFS), “immune-related duration of response” (irDR), are as defined and assessed by Immune-related response criteria (irRECIST, Nishino et. al. J Immunother Cancer 2014; 2:17) for patients with locally advanced or metastatic solid tumors other than patients with metastatic CRPC. The disclosure of Nishino et. al. J Immunother Cancer 2014; 2:17 is herein incorporated by reference in its entirety.

As used herein, “in combination with” refers to the administration of the MEK inhibitor or a pharmaceutically acceptable salt thereof, and a fluoropyrimidine-containing therapy concurrently, sequentially or intermittently as separate dosage.

The term “additive” is used to mean that the result of the combination of two components of the combination therapy is no greater than the sum of each compound, component or targeted agent individually. The term “additive” means that there is no improvement in the disease condition or disorder being treated over the use of each component individually.

The term “synergy” or “synergistic” is used herein to mean that the effect of the combination of the two therapeutic agents of the combination therapy is greater than the sum of the effect of each agent when administered alone. A “synergistic amount” or “synergistically effective amount” is an amount of the combination of the two combination partners that results in a synergistic effect, as “synergistic” is defined herein. Determining a synergistic interaction between two combination partners, the optimum range for the effect and absolute dose ranges of each component for the effect may be definitively measured by administration of the combination partners over different w/w (weight per weight) ratio ranges and doses to patients in need of treatment. However, the observation of synergy in in vitro models or in vivo models can be predictive of the effect in humans and other species and in vitro models or in vivo models exist, as described herein, to measure a synergistic effect and the results of such studies can also be used to predict effective dose and plasma concentration ratio ranges and the absolute doses and plasma concentrations required in humans and other species by the application of pharmacokinetic/pharmacodynamic methods. For example, art-accepted in vitro and animal models of cancers described herein are known in the art, and are described in the Examples. Exemplary synergistic effects includes, but are not limited to, enhanced therapeutic efficacy, decreased dosage at equal or increased level of efficacy, reduced or delayed development of drug resistance, and simultaneous enhancement or equal therapeutic actions and reduction of unwanted side effects.

For example, a synergistic ratio of two therapeutic agents can be identified by determining a synergistic effect in an art-accepted in vitro (e.g., cancer cell line) or in vivo (animal model) model of any of the cancers described herein. Non-limiting examples of cancer cell lines and in vivo animal models of the cancers described herein are described in the Examples. Additional examples of art-accepted cancer cell lines and in vivo animal models are known in the art.

In some embodiments, “synergistic effect” as used herein refers to combination of a MEK inhibitor or a pharmaceutically acceptable salt thereof, and a fluoropyrimidine-containing therapy producing an effect, for example, any of the beneficial or desired results including clinical results as described herein, for example slowing the symptomatic progression of biliary tract cancer, or symptoms thereof, which is greater than the sum of the effect observed when the MEK inhibitor and the anticancer therapy (e.g., fluorouracil-containing therapy) are administered alone.

In some embodiments, the methods provided herein can result in a 1% to 99% (e.g., 1% to 98%, 1% to 95%, 1% to 90%, 1 to 85%, 1 to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 2% to 99%, 2% to 90%, 2% to 85%, 2% to 80%, 2% to 75%, 2% to 70%, 2% to 65%, 2% to 60%, 2% to 55%, 2% to 50%, 2% to 45%, 2% to 40%, 2% to 35%, 2% to 30%, 2% to 25%, 2% to 20%, 2% to 15%, 2% to 10%, 2% to 5%, 4% to 99%, 4% to 95%, 4% to 90%, 4% to 85%, 4% to 80%, 4% to 75%, 4% to 70%, 4% to 65%, 4% to 60%, 4% to 55%, 4% to 50%, 4% to 45%, 4% to 40%, 4% to 35%, 4% to 30%, 4% to 25%, 4% to 20%, 4% to 15%, 4% to 10%, 6% to 99%, 6% to 95%, 6% to 90%, 6% to 85%, 6% to 80%, 6% to 75%, 6% to 70%, 6% to 65%, 6% to 60%, 6% to 55%, 6% to 50%, 6% to 45%, 6% to 40%, 6% to 35%, 6% to 30%, 6% to 25%, 6% to 20%, 6% to 15%, 6% to 10%, 8% to 99%, 8% to 95%, 8% to 90%, 8% to 85%, 8% to 80%, 8% to 75%, 8% to 70%, 8% to 65%, 8% to 60%, 8% to 55%, 8% to 50%, 8% to 45%, 8% to 40%, 8% to 35%, 8% to 30%, 8% to 25%, 8% to 20%, 8% to 15%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 10% to 15%, 15% to 99%, 15% to 95%, 15% to 90%, 15% to 85%, 15% to 80%, 15% to 75%, 15% to 70%, 15% to 65%, 15% to 60%, 15% to 55%, 15% to 50%, 15% to 55%, 15% to 50%, 15% to 45%, 15% to 40%, 15% to 35%, 15% to 30%, 15% to 25%, 15% to 20%, 20% to 99%, 20% to 95%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 25% to 99%, 25% to 95%, 25% to 90%, 25% to 85%, 25% to 80%, 25% to 75%, 25% to 70%, 25% to 65%, 25% to 60%, 25% to 55%, 25% to 50%, 25% to 45%, 25% to 40%, 25% to 35%, 25% to 30%, 30% to 99%, 30% to 95%, 30% to 90%, 30% to 85%, 30% to 80%, 30% to 75%, 30% to 70%, 30% to 65%, 30% to 60%, 30% to 55%, 30% to 50%, 30% to 45%, 30% to 40%, 30% to 35%, 35% to 99%, 35% to 95%, 35% to 90%, 35% to 85%, 35% to 80%, 35% to 75%, 35% to 70%, 35% to 65%, 35% to 60%, 35% to 55%, 35% to 50%, 35% to 45%, 35% to 40%, 40% to 99%, 40% to 95%, 40% to 90%, 40% to 85%, 40% to 80%, 40% to 75%, 40% to 70%, 40% to 65%, 40% to 60%, 40% to 55%, 40% to 60%, 40% to 55%, 40% to 50%, 40% to 45%, 45% to 99%, 45% to 95%, 45% to 95%, 45% to 90%, 45% to 85%, 45% to 80%, 45% to 75%, 45% to 70%, 45% to 65%, 45% to 60%, 45% to 55%, 45% to 50%, 50% to 99%, 50% to 95%, 50% to 90%, 50% to 85%, 50% to 80%, 50% to 75%, 50% to 70%, 50% to 65%, 50% to 60%, 50% to 55%, 55% to 99%, 55% to 95%, 55% to 90%, 55% to 85%, 55% to 80%, 55% to 75%, 55% to 70%, 55% to 65%, 55% to 60%, 60% to 99%, 60% to 95%, 60% to 90%, 60% to 85%, 60% to 80%, 60% to 75%, 60% to 70%, 60% to 65%, 65% to 99%, 60% to 95%, 60% to 90%, 60% to 85%, 60% to 80%, 60% to 75%, 60% to 70%, 60% to 65%, 70% to 99%, 70% to 95%, 70% to 90%, 70% to 85%, 70% to 80%, 70% to 75%, 75% to 99%, 75% to 95%, 75% to 90%, 75% to 85%, 75% to 80%, 80% to 99%, 80% to 95%, 80% to 90%, 80% to 85%, 85% to 99%, 85% to 95%, 85% to 90%, 90% to 99%, 90% to 95%, or 95% to 100%) reduction in the volume or size of one or more solid tumors (e.g., one or more biliary tract cancer tumors) in a patient following treatment with the combination therapy for a period of time between 1 day and 2 years (e.g., between 1 day and 22 months, between 1 day and 20 months, between 1 day and 18 months, between 1 day and 16 months, between 1 day and 14 months, between 1 day and 12 months, between 1 day and 10 months, between 1 day and 9 months, between 1 day and 8 months, between 1 day and 7 months, between 1 day and 6 months, between 1 day and 5 months, between 1 day and 4 months, between 1 day and 3 months, between 1 day and 2 months, between 1 day and 1 month, between one week and 2 years, between 1 week and 22 months, between 1 week and 20 months, between 1 week and 18 months, between 1 week and 16 months, between 1 week and 14 months, between 1 week and 12 months, between 1 week and 10 months, between 1 week and 9 months, between 1 week and 8 months, between 1 week and 7 months, between 1 week and 6 months, between 1 week and 5 months, between 1 week and 4 months, between 1 week and 3 months, between 1 week and 2 months, between 1 week and 1 month, between 2 weeks and 2 years, between 2 weeks and 22 months, between 2 weeks and 20 months, between 2 weeks and 18 months, between 2 weeks and 16 months, between 2 weeks and 14 months, between 2 weeks and 12 months, between 2 weeks and 10 months, between 2 weeks and 9 months, between 2 weeks and 8 months, between 2 weeks and 7 months, between 2 weeks and 6 months, between 2 weeks and 5 months, between 2 weeks and 4 months, between 2 weeks and 3 months, between 2 weeks and 2 months, between 2 weeks and 1 month, between 1 month and 2 years, between 1 month and 22 months, between 1 month and 20 months, between 1 month and 18 months, between 1 month and 16 months, between 1 month and 14 months, between 1 month and 12 months, between 1 month and 10 months, between 1 month and 9 months, between 1 month and 8 months, between 1 month and 7 months, between 1 month and 6 months, between 1 month and 6 months, between 1 month and 5 months, between 1 month and 4 months, between 1 month and 3 months, between 1 month and 2 months, between 2 months and 2 years, between 2 months and 22 months, between 2 months and 20 months, between 2 months and 18 months, between 2 months and 16 months, between 2 months and 14 months, between 2 months and 12 months, between 2 months and 10 months, between 2 months and 9 months, between 2 months and 8 months, between 2 months and 7 months, between 2 months and 6 months, or between 2 months and 5 months, between 2 months and 4 months, between 3 months and 2 years, between 3 months and 22 months, between 3 months and 20 months, between 3 months and 18 months, between 3 months and 16 months, between 3 months and 14 months, between 3 months and 12 months, between 3 months and 10 months, between 3 months and 8 months, between 3 months and 6 months, between 4 months and 2 years, between 4 months and 22 months, between 4 months and 20 months, between 4 months and 18 months, between 4 months and 16 months, between 4 months and 14 months, between 4 months and 12 months, between 4 months and 10 months, between 4 months and 8 months, between 4 months and 6 months, between 6 months and 2 years, between 6 months and 22 months, between 6 months and 20 months, between 6 months and 18 months, between 6 months and 16 months, between 6 months and 14 months, between 6 months and 12 months, between 6 months and 10 months, or between 6 months and 8 months) (e.g., as compared to the size of the one or more solid tumors in the patient prior to treatment).

In some embodiments, any of the methods described herein can provide for a 1% to 99% (e.g., 1% to 98%, 1% to 95%, 1% to 90%, 1 to 85%, 1 to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 2% to 99%, 2% to 90%, 2% to 85%, 2% to 80%, 2% to 75%, 2% to 70%, 2% to 65%, 2% to 60%, 2% to 55%, 2% to 50%, 2% to 45%, 2% to 40%, 2% to 35%, 2% to 30%, 2% to 25%, 2% to 20%, 2% to 15%, 2% to 10%, 2% to 5%, 4% to 99%, 4% to 95%, 4% to 90%, 4% to 85%, 4% to 80%, 4% to 75%, 4% to 70%, 4% to 65%, 4% to 60%, 4% to 55%, 4% to 50%, 4% to 45%, 4% to 40%, 4% to 35%, 4% to 30%, 4% to 25%, 4% to 20%, 4% to 15%, 4% to 10%, 6% to 99%, 6% to 95%, 6% to 90%, 6% to 85%, 6% to 80%, 6% to 75%, 6% to 70%, 6% to 65%, 6% to 60%, 6% to 55%, 6% to 50%, 6% to 45%, 6% to 40%, 6% to 35%, 6% to 30%, 6% to 25%, 6% to 20%, 6% to 15%, 6% to 10%, 8% to 99%, 8% to 95%, 8% to 90%, 8% to 85%, 8% to 80%, 8% to 75%, 8% to 70%, 8% to 65%, 8% to 60%, 8% to 55%, 8% to 50%, 8% to 45%, 8% to 40%, 8% to 35%, 8% to 30%, 8% to 25%, 8% to 20%, 8% to 15%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 10% to 15%, 15% to 99%, 15% to 95%, 15% to 90%, 15% to 85%, 15% to 80%, 15% to 75%, 15% to 70%, 15% to 65%, 15% to 60%, 15% to 55%, 15% to 50%, 15% to 55%, 15% to 50%, 15% to 45%, 15% to 40%, 15% to 35%, 15% to 30%, 15% to 25%, 15% to 20%, 20% to 99%, 20% to 95%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 25% to 99%, 25% to 95%, 25% to 90%, 25% to 85%, 25% to 80%, 25% to 75%, 25% to 70%, 25% to 65%, 25% to 60%, 25% to 55%, 25% to 50%, 25% to 45%, 25% to 40%, 25% to 35%, 25% to 30%, 30% to 99%, 30% to 95%, 30% to 90%, 30% to 85%, 30% to 80%, 30% to 75%, 30% to 70%, 30% to 65%, 30% to 60%, 30% to 55%, 30% to 50%, 30% to 45%, 30% to 40%, 30% to 35%, 35% to 99%, 35% to 95%, 35% to 90%, 35% to 85%, 35% to 80%, 35% to 75%, 35% to 70%, 35% to 65%, 35% to 60%, 35% to 55%, 35% to 50%, 35% to 45%, 35% to 40%, 40% to 99%, 40% to 95%, 40% to 90%, 40% to 85%, 40% to 80%, 40% to 75%, 40% to 70%, 40% to 65%, 40% to 60%, 40% to 55%, 40% to 60%, 40% to 55%, 40% to 50%, 40% to 45%, 45% to 99%, 45% to 95%, 45% to 95%, 45% to 90%, 45% to 85%, 45% to 80%, 45% to 75%, 45% to 70%, 45% to 65%, 45% to 60%, 45% to 55%, 45% to 50%, 50% to 99%, 50% to 95%, 50% to 90%, 50% to 85%, 50% to 80%, 50% to 75%, 50% to 70%, 50% to 65%, 50% to 60%, 50% to 55%, 55% to 99%, 55% to 95%, 55% to 90%, 55% to 85%, 55% to 80%, 55% to 75%, 55% to 70%, 55% to 65%, 55% to 60%, 60% to 99%, 60% to 95%, 60% to 90%, 60% to 85%, 60% to 80%, 60% to 75%, 60% to 70%, 60% to 65%, 65% to 99%, 60% to 95%, 60% to 90%, 60% to 85%, 60% to 80%, 60% to 75%, 60% to 70%, 60% to 65%, 70% to 99%, 70% to 95%, 70% to 90%, 70% to 85%, 70% to 80%, 70% to 75%, 75% to 99%, 75% to 95%, 75% to 90%, 75% to 85%, 75% to 80%, 80% to 99%, 80% to 95%, 80% to 90%, 80% to 85%, 85% to 99%, 85% to 95%, 85% to 90%, 90% to 99%, 90% to 95%, or 95% to 100%) reduction in the risk of developing a metastasis or the risk of developing an additional metastasis in a patient having a cancer (e.g., biliary tract cancer).

The phrase “time of survival” or “survival time” means the length of time between the identification or diagnosis of cancer (e.g., any of the cancers described herein) in a mammal by a medical professional and the time of death of the mammal (caused by the cancer). Methods of increasing the time of survival in a mammal having a cancer are described herein.

In some embodiments, any of the methods described herein can result in an increase (e.g., a 1% to 400%, 1% to 380%, 1% to 360%, 1% to 340%, 1% to 320%, 1% to 300%, 1% to 280%, 1% to 260%, 1% to 240%, 1% to 220%, 1% to 200%, 1% to 180%, 1% to 160%, 1% to 140%, 1% to 120%, 1% to 100%, 1% to 95%, 1% to 90%, 1% to 85%, 1% to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 5% to 400%, 5% to 380%, 5% to 360%, 5% to 340%, 5% to 320%, 5% to 300%, 5% to 280%, 5% to 260%, 5% to 240%, 5% to 220%, 5% to 200%, 5% to 180%, 5% to 160%, 5% to 140%, 5% to 120%, 5% to 100%, 5% to 90%, 5% to 80%, 5% to 70%, 5% to 60%, 5% to 50%, 5% to 40%, 5% to 30%, 5% to 20%, 5% to 10%, 10% to 400%, 10% to 380%, 10% to 360%, 10% to 340%, 10% to 320%, 10% to 300%, 10% to 280%, 10% to 260%, 10% to 240%, 10% to 220%, 10% to 200%, 10% to 180%, 10% to 160%, 10% to 140%, 10% to 120%, 10% to 100%, 10% to 90%, 10% to 80%, 10% to 70%, 10% to 60%, 10% to 50%, 10% to 40%, 10% to 30%, 10% to 20%, 20% to 400%, 20% to 380%, 20% to 360%, 20% to 340%, 20% to 320%, 20% to 300%, 20% to 280%, 20% to 260%, 20% to 240%, 20% to 220%, 20% to 200%, 20% to 180%, 20% to 160%, 20% to 140%, 20% to 120%, 20% to 100%, 20% to 90%, 20% to 80%, 20% to 70%, 20% to 60%, 20% to 50%, 20% to 40%, 20% to 30%, 30% to 400%, 30% to 380%, 30% to 360%, 30% to 340%, 30% to 320%, 30% to 300%, 30% to 280%, 30% to 260%, 30% to 240%, 30% to 220%, 30% to 200%, 30% to 180%, 30% to 160%, 30% to 140%, 30% to 120%, 30% to 100%, 30% to 90%, 30% to 80%, 30% to 70%, 30% to 60%, 30% to 50%, 30% to 40%, 40% to 400%, 40% to 380%, 40% to 360%, 40% to 340%, 40% to 320%, 40% to 300%, 40% to 280%, 40% to 260%, 40% to 240%, 40% to 220%, 40% to 200%, 40% to 180%, 40% to 160%, 40% to 140%, 40% to 120%, 40% to 100%, 40% to 90%, 40% to 80%, 40% to 70%, 40% to 60%, 40% to 50%, 50% to 400%, 50% to 380%, 50% to 360%, 50% to 340%, 50% to 320%, 50% to 300%, 50% to 280%, 50% to 260%, 50% to 240%, 50% to 220%, 50% to 200%, 50% to 180%, 50% to 160%, 50% to 140%, 50% to 140%, 50% to 120%, 50% to 100%, 50% to 90%, 50% to 80%, 50% to 70%, 50% to 60%, 60% to 400%, 60% to 380%, 60% to 360%, 60% to 340%, 60% to 320%, 60% to 300%, 60% to 280%, 60% to 260%, 60% to 240%, 60% to 220%, 60% to 200%, 60% to 180%, 60% to 160%, 60% to 140%, 60% to 120%, 60% to 100%, 60% to 90%, 60% to 80%, 60% to 70%, 70% to 400%, 70% to 380%, 70% to 360%, 70% to 340%, 70% to 320%, 70% to 300%, 70% to 280%, 70% to 260%, 70% to 240%, 70% to 220%, 70% to 200%, 70% to 180%, 70% to 160%, 70% to 140%, 70% to 120%, to 100%, 70% to 90%, 70% to 80%, 80% to 400%, 80% to 380%, 80% to 360%, 80% to 340%, 80% to 320%, 80% to 300%, 80% to 280%, 80% to 260%, 80% to 240%, 80% to 220%, 80% to 200%, 80% to 180%, 80% to 160%, 80% to 140%, 80% to 120%, 80% to 100%, 80% to 90%, 90% to 400%, 90% to 380%, 90% to 360%, 90% to 340%, 90% to 320%, 90% to 300%, 90% to 280%, 90% to 260%, 90% to 240%, 90% to 220%, 90% to 200%, 90% to 180%, 90% to 160%, 90% to 140%, 90% to 120%, 90% to 100%, 100% to 400%, 100% to 380%, 100% to 360%, 100% to 340%, 100% to 320%, 100% to 300%, 100% to 280%, 100% to 260%, 100% to 240%, 100% to 220%, 100% to 200%, 100% to 180%, 100% to 160%, 100% to 140%, 100% to 120%, 120% to 400%, 120% to 380%, 120% to 360%, 120% to 340%, 120% to 320%, 120% to 300%, 120% to 280%, 120% to 260%, 120% to 240%, 120% to 220%, 120% to 200%, 120% to 180%, 120% to 160%, 120% to 140%, 140% to 400%, 140% to 380%, 140% to 360%, 140% to 340%, 140% to 320%, 140% to 300%, 140% to 280%, 140% to 260%, 140% to 240%, 140% to 220%, 140% to 200%, 140% to 180%, 140% to 160%, 160% to 400%, 160% to 380%, 160% to 360%, 160% to 340%, 160% to 320%, 160% to 300%, 160% to 280%, 160% to 260%, 160% to 240%, 160% to 220%, 160% to 200%, 160% to 180%, 180% to 400%, 180% to 380%, 180% to 360%, 180% to 340%, 180% to 320%, 180% to 300%, 180% to 280%, 180% to 260%, 180% to 240%, 180% to 220%, 180% to 200%, 200% to 400%, 200% to 380%, 200% to 360%, 200% to 340%, 200% to 320%, 200% to 300%, 200% to 280%, 200% to 260%, 200% to 240%, 200% to 220%, 220% to 400%, 220% to 380%, 220% to 360%, 220% to 340%, 220% to 320%, 220% to 300%, 220% to 280%, 220% to 260%, 220% to 240%, 240% to 400%, 240% to 380%, 240% to 360%, 240% to 340%, 240% to 320%, 240% to 300%, 240% to 280%, 240% to 260%, 260% to 400%, 260% to 380%, 260% to 360%, 260% to 340%, 260% to 320%, 260% to 300%, 260% to 280%, 280% to 400%, 280% to 380%, 280% to 360%, 280% to 340%, 280% to 320%, 280% to 300%, 300% to 400%, 300% to 380%, 300% to 360%, 300% to 340%, or 300% to 320%) in the time of survival of the patient (e.g., as compared to a patient having a similar cancer (e.g., biliary tract cancer) and administered a different treatment or not receiving a treatment).

As used herein, the term “cytokine” refers generically to proteins released by one cell population that act on another cell as intercellular mediators or have an autocrine effect on the cells producing the proteins. Examples of such cytokines include lymphokines, monokines; interleukins (“ILs”) such as IL-1, IL-1a, IL-2, IL-3, IL-4, IL-5, IL-6, IL-7, IL-8, IL-9, IL10, IL-11, IL-12, IL-13, IL-15, IL-17A-F, IL-18 to IL-29 (such as IL-23), IL-31, including PROLEUKIN® rIL-2; a tumor-necrosis factor such as TNF-a or TNF-β, TGF-1-3; and other polypeptide factors including leukemia inhibitory factor (“LIF”), ciliary neurotrophic factor (“CNTF”), CNTF-like cytokine (“CLC”), cardiotrophin (“CT”), and kit ligand (“L”).

As used herein, the term “chemokine” refers to soluble factors (e.g., cytokines) that have the ability to selectively induce chemotaxis and activation of leukocytes. They also trigger processes of angiogenesis, inflammation, wound healing, and tumorigenesis. Example chemokines include IL-8, a human homolog of murine keratinocyte chemoattractant (KC).

Unless otherwise defined, all technical and scientific terms used herein have the same meaning as commonly understood by one of ordinary skill in the art to which this invention belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. As used herein, the singular form “a”, “an”, and “the” include plural references unless indicated otherwise. For example, “an” excipient includes one or more excipients. It is understood that aspects and variations of the invention described herein include “consisting of” and/or “consisting essentially of” aspects and variations. In some embodiments, methods consisting essentially of an administration step as disclosed herein include methods wherein a patient has failed a prior therapy, e.g., administration of one or more anticancer agents, e.g., administration of one or more anticancer agents independently selected from chemotherapeutic agents and targeted therapeutic agents (administered to the patient before the period of time) or has been refractory to such prior therapy, and/or wherein the cancer has metastasized or recurred. In some embodiments, methods consisting essentially of an administration step as disclosed herein include methods wherein a patient undergoes surgery, radiation, and/or other regimens prior to, substantially at the same time as, or following such an administration step as disclosed herein, and/or where the patient is administered other chemical and/or biological therapeutic agents following such an administration step as disclosed herein.

Exemplary methods and materials are described herein, although methods and materials similar or equivalent to those described herein can also be used in the practice or testing of the invention. The materials, methods, and examples are illustrative only and not intended to be limiting. All publications, patent applications, patents, sequences, database entries, and other references mentioned herein are incorporated by reference in their entirety.

### Methods, Uses, and Medicaments

In one embodiment, an amount of a MEK inhibitor, or a pharmaceutically acceptable salt thereof, is used in combination with an amount of a fluoropyrimidine-containing therapy, wherein the amounts together are effective in the treatment of cancer.

In one embodiment, a therapeutically effective amount of each of the combination partners of a combination therapy of the invention are administered separately and may be administered simultaneously, sequentially, or intermittently, and in any order, at specific or varying time intervals (e.g., during the period of time).

In one embodiment, provided herein is a method of treating biliary tract cancer, which comprises or consists essentially of administering, during the period of time, of a combination therapy consisting essentially of a MEK inhibitor or a pharmaceutically acceptable salt thereof, and a fluoropyrimidine-containing therapy, to a patient in need thereof, wherein the individual combination partners are administered in jointly therapeutically effective amounts (for example in synergistically effective amounts). The individual combination partners of a combination therapy of the invention may be administered in daily or intermittent dosages during the period of time. The individual combination partners of a combination therapy of the invention may be administered separately at different times and in any order during the period of time, or concurrently in divided combination forms during the period of time. In one embodiment, the MEK inhibitor or a pharmaceutically acceptable salt thereof is administered on a daily basis, either once daily or twice daily, during the period of time. In one embodiment, the MEK inhibitor or a pharmaceutically acceptable salt thereof is administered twice daily on a daily basis, during the period of time. In one embodiment, the anticancer therapy is administered on a daily basis, during the period of time. In one embodiment, the anticancer therapy administered twice a day, on a daily basis, during the period of time. The instant invention is therefore to be understood as embracing all such regimens of simultaneous or alternating treatment during the period of time and the term “administering” is to be interpreted accordingly.

The term “jointly therapeutically effective amount” as used herein means when the therapeutic agents of a combination described herein are given to the patient simultaneously or separately (e.g., in a chronologically staggered manner, for example a sequence-specific manner) in such time intervals that they show an interaction (e.g., a joint therapeutic effect, for example a synergistic effect). Whether this is the case can, inter alia, be determined by following the blood levels and showing that the combination components are present in the blood of the human to be treated at least during certain time intervals.

In one embodiment, provided herein is a method of treating a subject having biliary tract cancer comprising, consisting essentially of, or consisting of administering to said subject a combination therapy as described herein, during a period of time, in a quantity which is jointly therapeutically effective against biliary tract cancer. In one embodiment, the biliary tract cancer is advanced biliary tract cancer.

In some embodiments, the subject was previously treated, before the period of time, with one or more therapeutic agents, e.g., treatment with at least one anticancer treatment independently selected from chemotherapy (e.g., an analogue of deoxycytidine, e.g., Gemzar®, gemcitabine), targeted therapeutic agents (e.g., a MEK inhibitor, e.g., trametinib or binimetinib or a pharmaceutically acceptable salt thereof, as a monotherapy), radiation therapy, and surgery.

The term “chemotherapy” or “chemotherapeutic agent” as used herein refers to a chemotherapeutic agent, or a combination of two, three, four, or more chemotherapeutic agents, for the treatment of cancer. When a chemotherapy consists more than one chemotherapeutic agents, the chemotherapeutic agents can be administered to the patient on the same day or on different days in the same treatment cycle.

A “chemotherapeutic agent” is a chemical compound useful in the treatment of cancer. Examples of chemotherapeutic agents include alkylating agents such as thiotepa and cyclophosphamide (CYTOXAN®); alkyl sulfonates such as busulfan, improsulfan, and piposulfan; aziridines such as benzodopa, carboquone, meturedopa, and uredopa; ethylenimines and methylamelamines including altretamine, triethylenemelamine, triethylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; pemetrexed; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; TLK-286; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma I I and calicheamicin omegal 1 (see, e.g., Nicolaou et al., Angew. Chem Intl. Ed. Engl., 33: 183-186 (1994)); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HC1 liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and fluoropyrimidine-containing chemotherapies such as 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDIS1NE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel, also known as nab-paclitaxel (ABRAXANE™), and doxetaxel (TAXOTERE®); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluorometlhylomithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, FOLFOX, an abbreviation for a treatment regimen with oxaliplatin (ELOXATIN™) combined with 5-FU and leucovovin, FOLFOXIRI an abbreviation for a treatment regimen with irinotecan and oxaliplatin and 5-FU, and mFOLFOX-6, an abbreviation for a treatment regimen with leucovorin calcium (folinic acid), fluorouracil, and oxaliplatin.

Additional examples of chemotherapeutic agents include anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY117018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRF1) agonists such as leuprolide acetate (LUPRON® and ELIGARD®), goserelin acetate, buserelin acetate and tripterelin; anti-androgens such as fiutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RJVISOR®), letrozole (FEMARA®), and anastrozole (ARIMIDEX®). In addition, such definition of chemotherapeutic agents includes bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); as well as troxacitabine (a 1,3-dioxolane nucleoside cytosine analog); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); an anti-estrogen such as fulvestrant; irinotecan; rmRH (e.g., ABARELIX®); 17AAG (geldanamycin derivative that is a heat shock protein (Hsp) 90 poison), and pharmaceutically acceptable salts, acids or derivatives of any of the above.

A “platinum-based chemotherapy” as used herein, refers to a chemotherapy wherein at least one chemotherapeutic agent is a coordination complex of platinum. Exemplary platinum-based chemotherapy includes, without limitation, cisplatin, carboplatin, oxaliplatin, nedaplatin, gemcitabine in combination with cisplatin, carboplatin in combination with pemetremed.

A “targeted therapeutic agent” as used herein includes, refers to a molecule that blocks the growth of cancer cells by interfering with specific targeted molecules needed for carcinogenesis and tumor growth, rather than by simply interfering with all rapidly dividing cells (e.g. with traditional chemotherapy), and includes but is not limited to, receptor tyrosine kinase-targeted therapeutic agents (for example cabozantinib, crizotinib, erlotinib, gefitinib, imatinib, lapatinib, nilotinib, pazopanib, pertuzumab, regorafenib, sunitinib, and trastuzumab), signal transduction pathway inhibitors (for example, Ras-Raf-MEK-ERK pathway inhibitors (e.g. sorafenib, trametinib, vemurafenib, selumetinib, binimetinib, cobimetinib), PI3K-Akt-mTOR-S6K pathway inhibitors (e.g. everolimus, rapamycin, perifosine, temsirolimus) and modulators of the apoptosis pathway (e.g. obataclax)), anti-EGFR antibodies (e.g., cetuximab and panitumumab), anti-VEGF antibodies (e.g., bevacizumab), angiogenesis-targeted therapies (for example, aflibercept and bevacizumab). In one embodiment, the one or more therapeutic agents that were administered to the patient before the period of time is chemotherapy. In one embodiment, chemotherapy is selected from one or more of a platinum-based chemotherapy and a fluoropyrimidine-containing therapy. In one embodiment, the one or more therapeutic agents that were administered to the patient before the period of time is a Ras-Raf-MEK-ERK pathway inhibitor. In one embodiment, the therapeutic agent administered to the patient before the period of time is an EGFR kinase inhibitor. In one embodiments, the therapeutic agent administered to the patient before the period of time is a MEK inhibitor as a monotherapy or a fluoropyrimidine-containing therapy as a monotherapy. In one embodiment, the one or more therapeutic agents that were administered to the patient before the period of time is an anti-metabolite.

In some embodiments of any of the methods described herein, before the period of time, the patient was treated with a prior therapy, and optionally, the prior therapy was determined to be ineffective, and/or the patient was determined to be resistant to the prior therapy. In some embodiments of any of the methods described herein, before the period of time, the patient was treated with gemcitabine (e.g., as a monotherapy), and optionally, the previous administration of gemcitabine was determined to be ineffective, and/or the patient was determined to be resistant to gemcitabine. In some embodiments of any of the methods described herein, before the period of time, the patient was treated with gemcitabine and cisplatin, and optionally, the previous administration of gemcitabine and cisplatin was determined to be ineffective, and/or the patient was determined to be resistant to one or both of gemcitabine and cisplatin. In some embodiments of any of the methods described herein, before the period of time, the patient was treated with gemcitabine and oxaliplatin, and optionally, the previous administration of gemcitabine and oxaliplatin was determined to be ineffective, and/or the patient was determined to be resistant to one or both of gemcitabine and oxaliplatin. In some embodiments of any of the methods described herein, before the period of time, the patient was treated with 5-fluorouracil (e.g., as a monotherapy), and optionally, the previous administration of 5-fluorouracil was determined to be ineffective, and/or the patient was determined to be resistant to 5-fluorouracil. In some embodiments of any of the methods described herein, before the period of time, the patient was treated with 5-fluorouracil and leucovorin, and optionally, the previous administration of 5-fluorouracil and leucovorin was determined to be ineffective and/or the patient was determined to be resistant to one or both of 5-FU and leucovorin. In some embodiments, a subject has been administered 5-fluorouracil and cisplatin prior to the period of time, and optionally, the previous administration of 5-fluorouracil and cisplatin was determined to be ineffective and/or the patient was determined to be resistant to one or both of 5-fluorouracil and cisplatin. In some embodiments, a subject has been administered 5-fluorouracil, epirubicin, and cisplatin prior to the period of time, and optionally, the prior administration of 5-fluorouracil, epirubicin, and cisplatin was determined to be ineffective and/or the patient was determined to be resistant to one or more of 5-fluorouracil, epirubicin, and cisplatin. In some embodiments, a subject has been administered 5-fluorouracil and irinotecan prior to the period of time, and optionally, the prior administration of 5-fluorouracil and irinotecan was determined to be ineffective and/or the patient was determined to be resistant to one or both of 5-fluorouracil and irinotecan. In some embodiments of any of the methods described herein, before the period of time, the patient was treated with capecitabine (e.g., as a monotherapy), and optionally, the previous administration of capecitabine was determined to be ineffective and/or the patient was determined to be resistant to capecitabine. In some embodiments of any of the methods described herein, before the period of time, the patient was treated with gemcitabine and optionally, the previous administration of gemcitabine was determined to be ineffective and/or the patient was determined to be resistant to gemcitabine. In some embodiments of any of the methods described herein, before the period of time, the patient was treated with gemcitabine and oxaliplatin, and optionally, the previous administration of gemcitabine and oxaliplatin was determined to be ineffective and/or the patient was determined to be resistant to one or more of gemcitabine and oxaliplatin. In some embodiments of any of the methods described herein, before the period of time, the patient was treated with lapatinib, and optionally, the previous administration of lapatinib was determined to be ineffective and/or the patient was determined to be resistant to lapatinib. In some embodiments of any of the methods described herein, before the period of time, the patient was treated with erlotinib, and optionally, the previous administration of erlotinib was determined to be ineffective and/or the patient was determined to be resistant to erlotinib. In some embodiments of any of the methods described herein, before the period of time, the patient was treated with cetuximab or panitumumab, and optionally, the previous administration of cetuximab or panitumumab was determined to be ineffective and/or the patient was determined to be resistant to one or both of cetuximab and panitumumab. In some embodiments of any of the methods described herein, before the period of time, the patient was treated with bevacizumab (e.g., as a monotherapy), and optionally, the previous administration of bevacizumab was determined to be ineffective and/or the patient was determined to be resistant to bevacizumab. In some embodiments of any of the methods described herein, before the period of time, the patient was treated with bevacizumab and mFOLFOX6, and optionally, the previous administration of bevacizumab and mFOLFOX6 was determined to be ineffective and/or the patient was determined to be resistant to one or both of bevacizumab and mFOLFOX6. In some embodiments of any of the methods described herein, before the period of time, the patient was treated with bevacizumab in combination with gemcitabine and capecitabine, and optionally, the previous administration of bevacizumab in combination with gemcitabine and capecitabine was determined to be ineffective and/or the patient was determined to be resistant to one or more of bevacizumab, gemcitabine, and capecitabine. In some embodiments of any of the methods described herein, before the period of time, the patient was treated with a MEK inhibitor (e.g., as a monotherapy) and optionally, the previous administration of the MEK inhibitor was determined to be ineffective and/or the patient was determined to be resistant to the MEK inhibitor. In some embodiments of any of the methods described herein, before the period of time, the patient was treated with selumetinib, and optionally, the previous treatment with selumetinib was determined to be ineffective and/or the patient was determined to be resistant to selumetinib. In some embodiments of any of the methods described herein, before the period of time, the patient was treated with trametinib, and optionally, the previous treatment with trametinib was determined to be resistant to trametinib. In some embodiments of any of the methods described herein, before the period of time, the patient was treated with binimetinib, and optionally, the previous treatment with binimetinib was determined to be ineffective and/or the patient was determined to be resistant to binimetinib. In some embodiments of any of the methods described herein, the one or more therapeutic agents that were administered to the patient before the period of time was unsuccessful or ineffective (e.g., therapeutically unsuccessful or ineffective as determined by a physician).

In one embodiment, the patient has been administered surgery before the period of time. Non-limiting examples of surgery include, e.g., open surgery or minimally invasive surgery. Surgery can include, e.g., removing an entire tumor, debulking of a tumor, or removing a tumor that is causing pain or pressure in the subject. Methods for performing open surgery and minimally invasive surgery on a subject having a cancer are known in the art.

In one embodiment, the patient has received radiotherapy before the period of time. Non-limiting examples of radiation therapy include external radiation beam therapy (e.g., external beam therapy using kilovoltage X-rays or megavoltage X-rays) or internal radiation therapy. Internal radiation therapy (also called brachytherapy) can include the use of, e.g., low-dose internal radiation therapy or high-dose internal radiation therapy. Low-dose internal radiation therapy includes, e.g., inserting small radioactive pellets (also called seeds) into or proximal to a cancer tissue in the subject. High-dose internal radiation therapy includes, e.g., inserting a thin tube (e.g., a catheter) or an implant into or proximal to a cancer tissue in the subject, and delivering a high dose of radiation to the thin tube or implant using a radiation machine. Methods for performing radiation therapy on a subject having a cancer are known in the art.

In one embodiment, the MEK inhibitor is binimetinib or a pharmaceutically acceptable salt thereof. In one embodiment, the MEK inhibitor is binimetinib as the free base. In one embodiment, the MEK inhibitor is a pharmaceutically acceptable salt of binimetinib. In one embodiment, the MEK inhibitor is crystallized binimetinib. In one embodiment, binimetinib is orally administered during the period of time. In one embodiment, binimetinib is administered as a tablet during the period of time. In one embodiment, a tablet formulation of binimetinib comprises about 5 mg to about 50 mg (e.g., 5 mg to about 45 mg, about 5 mg to about 40 mg, about 5 mg to about 35 mg, about 5 mg to about 30 mg, about 5 mg to about 25 mg, about 5 mg to about 20 mg, about 5 mg to about 18 mg, about 5 mg to about 16 mg, about 5 mg to about 14 mg, about 5 mg to about 12 mg, about 5 mg to about 10 mg, about 5 mg to about 8 mg, about 10 mg to about 50 mg, about 10 mg to about 45 mg, about 10 mg to about 40 mg, about 10 mg to about 35 mg, about 10 mg to about 30 mg, about 10 mg to about 25 mg, about 10 mg to about 20 mg, about 10 mg to about 18 mg, about 10 mg to about 16 mg, about 10 mg to about 14 mg, about 10 mg to about 12 mg, about 12 mg to about 50 mg, about 12 mg to about 45 mg, about 12 mg to about 45 mg, about 12 mg to about 40 mg, about 12 mg to about 35 mg, about 12 mg to about 30 mg, about 12 mg to about 25 mg, about 12 mg to about 20 mg, about 12 mg to about 18 mg, about 12 mg to about 16 mg, about 12 mg to about 14 mg, about 14 mg to about 50 mg, about 14 mg to about 45 mg, about 14 mg to about 40 mg, about 14 mg to about 35 mg, about 14 mg to about 30 mg, about 14 mg to about 25 mg, about 14 mg to about 20 mg, about 14 mg to about 18 mg, about 14 mg to about 16 mg, about 16 mg to about 50 mg, about 16 mg to about 45 mg, about 16 mg to about 40 mg, about 16 mg to about 35 mg, about 16 mg to about 30 mg, about 16 mg to about 25 mg, about 16 mg to about 20 mg, about 16 mg to about 18 mg, about 18 mg to about 50 mg, about 18 mg to about 45 mg, about 18 mg to about 40 mg, about 18 mg to about 35 mg, about 18 mg to about 30 mg, about 18 mg to about 25 mg, about 18 mg to about 20 mg, about 20 mg to about 50 mg, about 20 mg to about 45 mg, about 20 mg to about 40 mg, about 20 mg to about 35 mg, about 20 mg to about 30 mg, about 20 mg to about 25 mg, about 25 mg to about 50 mg, about 25 mg to about 45 mg, about 25 mg to about 40 mg, about 25 mg to about 35 mg, about 25 mg to about 30 mg, about 30 mg to about 50 mg, about 30 mg to about 45 mg, about 30 mg to about 40 mg, about 30 mg to about 35 mg, about 35 mg to about 50 mg, about 35 mg to about 45 mg, about 35 mg to about 40 mg, about 40 mg to about 50 mg, about 40 mg to about 45 mg, about 45 mg to about 50 mg, about 5 mg, about 10 mg, about 15 mg, about 20 mg, about 25 mg, about 30 mg, about 35 mg, about 40 mg, about 45 mg, or about 50 mg) of binimetinib or a pharmaceutically acceptable salt thereof. In one embodiment, a tablet formulation of binimetinib comprises about 5 mg to about 50 mg (e.g., any of the subranges or values within this range described herein, e.g., about 15 mg) of crystallized binimetinib. In one embodiment, binimetinib is orally administered twice daily during the period of time. In one embodiment, binimetinib is orally administered twice daily during the period of time, wherein the second dose of binimetinib is administered about 1 hour, about 2 hours, about 3 hours, about 4 hours, about 5 hours, about 6 hours, about 7 hours, about 8 hours, about 9 hours, about 10 hours, about 11 hours, or about 12 hours (e.g., 12 hours±2 hours) after the first dose of binimetinib during the period of time. In one embodiment, binimetinib is orally administered daily in the amount of about 10 mg to about 100 mg (e.g., about 10 mg to about 95 mg, about 10 mg to about 90 mg, about 10 mg to about 85 mg, about 10 mg to about 80 mg, about 10 mg to about 75 mg, about 10 mg to about 70 mg, about 10 mg to about 65 mg, about 10 mg to about 60 mg, about 10 mg to about 55 mg, about 10 mg to about 50 mg, about 10 mg to about 45 mg, about 10 mg to about 40 mg, about 10 mg to about 35 mg, about 10 mg to about 30 mg, about 10 mg to about 25 mg, about 10 mg to about 20 mg, about 10 mg to about 15 mg, about 15 mg to about 100 mg, about 15 mg to about 95 mg, about 15 mg to about 90 mg, about 15 mg to about 85 mg, about 15 mg to about 80 mg, about 15 mg to about 75 mg, about 15 mg to about 70 mg, about 15 mg to about 65 mg, about 15 mg to about 60 mg, about 15 mg to about 55 mg, about 15 mg to about 50 mg, about 15 mg to about 45 mg, about 15 mg to about 40 mg, about 15 mg to about 35 mg, about 15 mg to about 30 mg, about 15 mg to about 25 mg, about 15 mg to about 20 mg, about 20 mg to about 100 mg, about 20 mg to about 95 mg, about 20 mg to about 90 mg, about 20 mg to about 85 mg, about 20 mg to about 80 mg, about 20 mg to about 75 mg, about 20 mg to about 70 mg, about 20 mg to about 65 mg, about 20 mg to about 60 mg, about 20 mg to about 55 mg, about 20 mg to about 50 mg, about 20 mg to about 45 mg, about 20 mg to about 40 mg, about 20 mg to about 35 mg, about 20 mg to about 30 mg, about 20 mg to about 25 mg, about 25 mg to about 100 mg, about 25 mg to about 95 mg, about 25 mg to about 90 mg, about 25 mg to about 85 mg, about 25 mg to about 80 mg, about 25 mg to about 75 mg, about 25 mg to about 70 mg, about 25 mg to about 65 mg, about 25 mg to about 60 mg, about 25 mg to about 55 mg, about 25 mg to about 50 mg, about 25 mg to about 45 mg, about 25 mg to about 40 mg, about 25 mg to about 35 mg, about 25 mg to about 30 mg, about 30 mg to about 100 mg, about 30 mg to about 95 mg, about 30 mg to about 90 mg, about 30 mg to about 85 mg, about 30 mg to about 80 mg, about 30 mg to about 75 mg, about 30 mg to about 70 mg, about 30 mg to about 65 mg, about 30 mg to about 60 mg, about 30 mg to about 55 mg, about 30 mg to about 50 mg, about 30 mg to about 45 mg, about 30 mg to about 40 mg, about 30 mg to about 35 mg, about 35 mg to about 100 mg, about 35 mg to about 95 mg, about 35 mg to about 90 mg, about 35 mg to about 85 mg, about 35 mg to about 80 mg, about 35 mg to about 75 mg, about 35 mg to about 70 mg, about 35 mg to about 65 mg, about 35 mg to about 60 mg, about 35 mg to about 55 mg, about 35 mg to about 50 mg, about 35 mg to about 45 mg, about 35 mg to about 40 mg, about 40 mg to about 100 mg, about 40 mg to about 95 mg, about 40 mg to about 90 mg, about 40 mg to about 85 mg, about 40 mg to about 80 mg, about 40 mg to about 75 mg, about 40 mg to about 70 mg, about 40 mg to about 65 mg, about 40 mg to about 60 mg, about 40 mg to about 55 mg, about 40 mg to about 50 mg, about 40 mg to about 45 mg, about 45 mg to about 100 mg, about 45 mg to about 95 mg, about 45 mg to about 90 mg, about 45 mg to about 85 mg, about 45 mg to about 80 mg, about 45 mg to about 75 mg, about 45 mg to about 70 mg, about 45 mg to about 65 mg, about 45 mg to about 60 mg, about 45 mg to about 55 mg, about 45 mg to about 50 mg, about 50 mg to about 100 mg, about 50 mg to about 95 mg, about 50 mg to about 90 mg, about 50 mg to about 85 mg, about 50 mg to about 80 mg, about 50 mg to about 75 mg, about 50 mg to about 70 mg, about 50 mg to about 65 mg, about 50 mg to about 60 mg, about 50 mg to about 55 mg, about 55 mg to about 100 mg, about 55 mg to about 95 mg, about 55 mg to about 90 mg, about 55 mg to about 85 mg, about 55 mg to about 80 mg, about 55 mg to about 75 mg, about 55 mg to about 70 mg, about 55 mg to about 65 mg, about 55 mg to about 60 mg, about 60 mg to about 100 mg, about 60 mg to about 95 mg, about 60 mg to about 90 mg, about 60 mg to about 85 mg, about 60 mg to about 80 mg, about 60 mg to about 75 mg, about 60 mg to about 70 mg, about 60 mg to about 65 mg, about 65 mg to about 100 mg, about 65 mg to about 95 mg, about 65 mg to about 90 mg, about 65 mg to about 85 mg, about 65 mg to about 80 mg, about 65 mg to about 75 mg, about 65 mg to about 70 mg, about 70 mg to about 100 mg, about 70 mg to about 95 mg, about 70 mg to about 90 mg, about 70 mg to about 85 mg, about 70 mg to about 80 mg, about 70 mg to about 75 mg, about 75 mg to about 100 mg, about 75 mg to about 95 mg, about 75 mg to about 90 mg, about 75 mg to about 85 mg, about 75 mg to about 80 mg, about 80 mg to about 100 mg, about 80 mg to about 95 mg, about 80 mg to about 90 mg, about 80 mg to about 85 mg, about 85 mg to about 100 mg, about 85 mg to about 95 mg, about 85 mg to about 90 mg, about 90 mg to about 100 mg, about 90 mg to about 95 mg, about 95 mg to about 100 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg) twice daily, during the period of time. In one embodiment, 15 mg of binimetinib is orally administered twice daily, during the period of time. In one embodiment, 30 mg of binimetinib is orally administered twice daily, during the period of time. In one embodiment, 45 mg of binimetinib is orally administered twice daily, during the period of time. In one embodiment, binimetinib is orally administered daily in the amount of about 10 mg to about 100 mg (e.g., any of the subranges or values in this range described herein, e.g., about 15 mg or about 30 mg or about 45 mg) BID for three weeks, followed by one week, two weeks, or three weeks without administration of binimetinib in at least one treatment cycle of three weeks, during the period of time. In one embodiment, binimetinib is orally administered twice daily in the amount of about 15 mg for three weeks followed by one week without administration of binimetinib in at least one treatment cycle of three weeks, during the period of time. In one embodiment, binimetinib is orally administered twice daily in the amount of about 30 mg for three weeks followed by one week without administration of binimetinib in at least one treatment cycle of three weeks, during the period of time. In one embodiment, binimetinib is orally administered twice daily in the amount of about 45 mg for three weeks followed by one week without administration of binimetinib in at least one treatment cycle of three weeks, during the period of time. In one embodiment, 45 mg of binimetinib is orally administered twice daily until observation of adverse effects, after which 30 mg of binimetinib is administered twice daily, during the period of time. In one embodiment, patients who have been dose reduced to 30 mg twice daily may re-escalate to 45 mg twice daily if the adverse effects that resulted in a dose reduction improve to baseline and remain stable for, e.g., up to 14 days, or up to three weeks, or up to 4 weeks, provided there are no other concomitant toxicities related to binimetinib that would prevent drug re-escalation, during the period of time.

In some embodiments, fluoropyrimidine-containing therapy is capecitabine. In one embodiment, capecitabine is orally administered, during the period of time. In one embodiment, capecitabine is administered as a tablet or a capsule during the period of time. In some embodiments, a table or capsule formulation of capecitabine comprises, e.g., about 10 mg to about 500 mg, about 10 mg to about 475 mg, about 10 mg to about 450 mg, about 10 mg to about 425 mg, about 10 mg to about 400 mg, about 10 mg to about 375 mg, about 10 mg to about 350 mg, about 10 mg to about 325 mg, about 10 mg to about 300 mg, about 10 mg to about 275 mg, about 10 mg to about 250 mg, about 10 mg to about 225 mg, about 10 mg to about 200 mg, about 10 mg to about 175 mg, about 10 mg to about 150 mg, about 10 mg to about 125 mg, about 10 mg to about 100 mg, about 10 mg to about 75 mg, about 10 mg to about 50 mg, about 10 mg to about 25 mg, about 25 mg to about 500 mg, about 25 mg to about 475 mg, about 25 mg to about 450 mg, about 25 mg to about 425 mg, about 25 mg to about 400 mg, about 25 mg to about 375 mg, about 25 mg to about 350 mg, about 25 mg to about 325 mg, about 25 mg to about 300 mg, about 25 mg to about 275 mg, about 25 mg to about 250 mg, about 25 mg to about 225 mg, about 25 mg to about 200 mg, about 25 mg to about 175 mg, about 25 mg to about 150 mg, about 25 mg to about 125 mg, about 25 mg to about 100 mg, about 25 mg to about 75 mg, about 25 mg to about 50 mg, about 50 mg to about 500 mg, about 50 mg to about 475 mg, about 50 mg to about 450 mg, about 50 mg to about 425 mg, about 50 mg to about 400 mg, about 50 mg to about 375 mg, about 50 mg to about 350 mg, about 50 mg to about 325 mg, about 50 mg to about 300 mg, about 50 mg to about 275 mg, about 50 mg to about 250 mg, about 50 mg to about 225 mg, about 50 mg to about 200 mg, about 50 mg to about 175 mg, about 50 mg to about 150 mg, about 50 mg to about 125 mg, about 50 mg to about 100 mg, about 50 mg to about 75 mg, about 75 mg to about 500 mg, about 75 mg to about 475 mg, about 75 mg to about 450 mg, about 75 mg to about 425 mg, about 75 mg to about 400 mg, about 75 mg to about 375 mg, about 75 mg to about 350 mg, about 75 mg to about 325 mg, about 75 mg to about 300 mg, about 75 mg to about 275 mg, about 75 mg to about 250 mg, about 75 mg to about 225 mg, about 75 mg to about 200 mg, about 75 mg to about 175 mg, about 75 mg to about 150 mg, about 75 mg to about 125 mg, about 75 mg to about 100 mg, about 100 mg to about 500 mg, about 100 mg to about 475 mg, about 100 mg to about 450 mg, about 100 mg to about 425 mg, about 100 mg to about 400 mg, about 100 mg to about 375 mg, about 100 mg to about 350 mg, about 100 mg to about 325 mg, about 100 mg to about 300 mg, about 100 mg to about 275 mg, about 100 mg to about 250 mg, about 100 mg to about 225 mg, about 100 mg to about 200 mg, about 100 mg to about 175 mg, about 100 mg to about 150 mg, about 100 mg to about 125 mg, about 125 mg to about 500 mg, about 125 mg to about 475 mg, about 125 mg to about 450 mg, about 125 mg to about 425 mg, about 125 mg to about 400 mg, about 125 mg to about 375 mg, about 125 mg to about 350 mg, about 125 mg to about 325 mg, about 125 mg to about 300 mg, about 125 mg to about 275 mg, about 125 mg to about 250 mg, about 125 mg to about 225 mg, about 125 mg to about 200 mg, about 125 mg to about 175 mg, about 125 mg to about 150 mg, about 150 mg to about 500 mg, about 150 mg to about 475 mg, about 150 mg to about 450 mg, about 150 mg to about 425 mg, about 150 mg to about 400 mg, about 150 mg to about 375 mg, about 150 mg to about 350 mg, about 150 mg to about 325 mg, about 150 mg to about 300 mg, about 150 mg to about 275 mg, about 150 mg to about 250 mg, about 150 mg to about 225 mg, about 150 mg to about 200 mg, about 150 mg to about 175 mg, about 175 mg to about 500 mg, about 175 mg to about 475 mg, about 175 mg to about 450 mg, about 175 mg to about 425 mg, about 175 mg to about 400 mg, about 175 mg to about 375 mg, about 175 mg to about 350 mg, about 175 mg to about 325 mg, about 175 mg to about 300 mg, about 175 mg to about 275 mg, about 175 mg to about 250 mg, about 175 mg to about 225 mg, about 175 mg to about 200 mg, about 200 mg to about 500 mg, about 200 mg to about 475 mg, about 200 mg to about 450 mg, about 200 mg to about 425 mg, about 200 mg to about 400 mg, about 200 mg to about 375 mg, about 200 mg to about 350 mg, about 200 mg to about 325 mg, about 200 mg to about 300 mg, about 200 mg to about 275 mg, about 200 mg to about 250 mg, about 200 mg to about 225 mg, about 225 mg to about 500 mg, about 225 mg to about 475 mg, about 225 mg to about 450 mg, about 225 mg to about 425 mg, about 225 mg to about 400 mg, about 225 mg to about 375 mg, about 225 mg to about 350 mg, about 225 mg to about 325 mg, about 225 mg to about 300 mg, about 225 mg to about 275 mg, about 225 mg to about 250 mg, about 250 mg to about 500 mg, about 250 mg to about 475 mg, about 250 mg to about 450 mg, about 250 mg to about 425 mg, about 250 mg to about 400 mg, about 250 mg to about 375 mg, about 250 mg to about 350 mg, about 250 mg to about 325 mg, about 250 mg to about 300 mg, about 250 mg to about 275 mg, about 275 mg to about 500 mg, about 275 mg to about 475 mg, about 275 mg to about 450 mg, about 275 mg to about 425 mg, about 275 mg to about 400 mg, about 275 mg to about 375 mg, about 275 mg to about 350 mg, about 275 mg to about 325 mg, about 275 mg to about 300 mg, about 300 mg to about 500 mg, about 300 mg to about 475 mg, about 300 mg to about 450 mg, about 300 mg to about 425 mg, about 300 mg to about 400 mg, about 300 mg to about 375 mg, about 300 mg to about 350 mg, about 300 mg to about 325 mg, about 325 mg to about 500 mg, about 325 mg to about 475 mg, about 325 mg to about 450 mg, about 325 mg to about 425 mg, about 325 mg to about 400 mg, about 325 mg to about 375 mg, about 325 mg to about 350 mg, about 350 mg to about 500 mg, about 350 mg to about 475 mg, about 350 mg to about 450 mg, about 350 mg to about 425 mg, about 350 mg to about 400 mg, about 350 mg to about 375 mg, about 375 mg to about 500 mg, about 375 mg to about 475 mg, about 375 mg to about 450 mg, about 375 mg to about 425 mg, about 375 mg to about 400 mg, about 400 mg to about 500 mg, about 400 mg to about 475 mg, about 400 mg to about 450 mg, about 400 mg to about 425 mg, about 425 mg to about 500 mg, about 425 mg to about 475 mg, about 425 mg to about 450 mg, about 450 mg to about 500 mg, about 450 mg to about 475 mg, or about 475 mg to about 500 mg of capecitabine.

In one embodiment, a tablet formulation of capecitabine comprises about 150 mg of capecitabine. In one embodiment, a tablet formulation of capecitabine comprises about 300 mg of capecitabine. In one embodiment, capecitabine is orally administered once, twice, or three times daily during the period of time. In one embodiment, capecitabine is orally administered twice daily during the period of time, wherein the second dose of capecitabine is administered about 1 hour, about 2 hours, about 3 hours, about 4 hours, about 5 hours, about 6 hours, about 7 hours, about 8 hours, about 9 hours, about 10 hours, about 11 hours, or about 12 hours (e.g., 12 hours±2 hours) after the first dose of capecitabine during the period of time. In one embodiment, capecitabine is orally administered twice daily during the period of time, wherein each administration of capecitabine occurs at least 30 minutes after a meal. In one embodiment, capecitabine is orally administered twice daily for 2 weeks followed by a one week rest period in a 3-week cycle. In one embodiment, capecitabine is administered orally at a dose of about 800 mg/m², or about 825 mg/m², or about 950 mg/m², or about 1000 mg/m², or about 1250 mg/m² twice daily. In one embodiment, capecitabine is administered orally at a dose of about 800 mg/m² twice daily during the period of time. In one embodiment, capecitabine is administered orally at a dose of about 825 mg/m² twice daily during the period of time. In one embodiment, capecitabine is administered orally at a dose of about 950 mg/m² twice daily during the period of time. In one embodiment, capecitabine is administered orally at a dose of about 1000 mg/m² twice daily during the period of time. In one embodiment, capecitabine is administered orally at a dose of about 1250 mg/m² twice daily during the period of time. In one embodiment, when capecitabine is administered orally at a dose of about 1250 mg/m² twice daily during the period of time, capecitabine may be administered according to the following table:

| Dose Level 1250 mg/m² | | | |
|---|---|---|---|
| Twice a Day | | Number of Tablets to | |
| Surface | Total Daily | be Taken at Each Dose | |
| Area | Dose* | (Morning and Evening) | |
| (m²) | (mg) | 150 mg | 500 mg |
| ≤1.25 | 3000 | 0 | 3 |
| 1.26-.37 | 3300 | 1 | 3 |
| 1.38-1.51 | 3600 | 2 | 3 |
| 1.52-1.65 | 4000 | 0 | 4 |
| 1.66-1.77 | 4300 | 1 | 4 |
| 1.78-1.91 | 4600 | 2 | 4 |
| 1.92-2.05 | 5000 | 0 | 5 |
| 2.06-2.17 | 5300 | 1 | 5 |
| ≥2.18 | 5600 | 2 | 5 |

In one embodiment, the invention provides a method for treating biliary tract cancer comprising or consisting essentially of administering to a patient in need thereof, during a period of time, a combination therapy comprising or consisting essentially of or consisting of therapeutically effective amounts, independently or in combination, of a MEK inhibitor and a fluoropyrimidine-containing therapy, wherein the MEK inhibitor is binimetinib or a pharmaceutically acceptable salt thereof. In one embodiment, the MEK inhibitor is binimetinib as the free base. In one embodiment, the MEK inhibitor is crystallized binimetinib. In one embodiment, binimetinib is orally administered twice daily in the amount of about 10 mg to about 100 mg (e.g., any of the subranges or values in this range described herein, e.g., about 30 mg or about 45 mg), during the period of time. In one embodiment, binimetinib is administered twice daily for two weeks followed by a one week rest period for a three week period. In one embodiment, the patient was previously treated with one or more prior therapies, e.g., one or more therapeutic agents, e.g., at least one treatment with another anticancer treatment, before the period of time.

In one embodiment, a method for treating biliary tract cancer comprises or consists essentially of administering to a patient in need thereof, during the period of time, a combination therapy consisting essentially of or consisting of therapeutically effective amounts, independently or in combination, of (a) a MEK inhibitor and (b) a fluoropyrimidine-containing therapy which is capecitabine, wherein capecitabine is administered orally twice daily in the amount of about 100 mg/m² to about 1250 mg/m² (e.g., about 100 mg/m² to about 1200 mg/m², about 100 mg/m² to about 1100 mg/m², about 100 mg/m² to about 1000 mg/m², about 100 mg/m² to about 900 mg/m², about 100 mg/m² to about 800 mg/m², about 100 mg/m² to about 700 mg/m², about 100 mg/m² to about 600 mg/m², about 100 mg/m² to about 500 mg/m², about 100 mg/m² to about 400 mg/m², about 100 mg/m² to about 300 mg/m², about 100 mg/m² to about 200 mg/m², about 200 mg/m² to about 1250 mg/m², about 200 mg/m² to about 1200 mg/m², about 200 mg/m² to about 1100 mg/m², about 200 mg/m² to about 1000 mg/m², about 200 mg/m² to about 900 mg/m², about 200 mg/m² to about 800 mg/m², about 200 mg/m² to about 700 mg/m², about 200 mg/m² to about 600 mg/m², about 200 mg/m² to about 500 mg/m², about 200 mg/m² to about 400 mg/m², about 200 mg/m² to about 300 mg/m², about 300 mg/m² to about 1250 mg/m², about 300 mg/m² to about 1200 mg/m², about 300 mg/m² to about 1100 mg/m², about 300 mg/m² to about 1000 mg/m², about 300 mg/m² to about 900 mg/m², about 300 mg/m² to about 800 mg/m², about 300 mg/m² to about 700 mg/m², about 300 mg/m² to about 600 mg/m², about 300 mg/m² to about 500 mg/m², about 300 mg/m² to about 400 mg/m², about 400 mg/m² to about 1250 mg/m², about 400 mg/m² to about 1200 mg/m², about 400 mg/m² to about 1100 mg/m², about 400 mg/m² to about 1000 mg/m², about 400 mg/m² to about 900 mg/m², about 400 mg/m² to about 800 mg/m², about 400 mg/m² to about 700 mg/m², about 400 mg/m² to about 600 mg/m², about 400 mg/m² to about 500 mg/m², about 500 mg/m² to about 1250 mg/m², about 500 mg/m² to about 1200 mg/m², about 500 mg/m² to about 1100 mg/m², about 500 mg/m² to about 1000 mg/m², about 500 mg/m² to about 900 mg/m², about 500 mg/m² to about 800 mg/m², about 500 mg/m² to about 700 mg/m², about 500 mg/m² to about 600 mg/m², about 600 mg/m² to about 1250 mg/m², about 600 mg/m² to about 1200 mg/m², about 600 mg/m² to about 1100 mg/m², about 600 mg/m² to about 1000 mg/m², about 600 mg/m² to about 900 mg/m², about 600 mg/m² to about 800 mg/m², about 600 mg/m² to about 700 mg/m², about 700 mg/m² to about 1250 mg/m², about 700 mg/m² to about 1200 mg/m², about 700 mg/m² to about 1100 mg/m², about 700 mg/m² to about 1000 mg/m², about 700 mg/m² to about 900 mg/m², about 700 mg/m² to about 800 mg/m², about 800 mg/m² to about 1250 mg/m², about 800 mg/m² to about 1200 mg/m², about 800 mg/m² to about 1100 mg/m², about 800 mg/m² to about 1000 mg/m², about 800 mg/m² to about 900 mg/m², about 900 mg/m² to about 1250 mg/m², about 900 mg/m² to about 1200 mg/m², about 900 mg/m² to about 1100 mg/m², about 900 mg/m² to about 1000 mg/m², about 1000 mg/m² to about 1250 mg/m², about 1000 mg/m² to about 1200 mg/m², about 1000 mg/m² to about 1100 mg/m², about 1100 mg/m² to about 1250 mg/m², or about 1100 mg/m² to about 1200 mg/m²) during the period of time. In one embodiment, capecitabine is administered orally twice daily in the amount of about 1000 mg/m² or about 1250 mg/m². In one embodiment, capecitabine is administered twice daily for two weeks followed by a one week rest period for a three week period. In one embodiment, the patient was previously treated with one or more prior therapies, e.g., one or more therapeutic agents, e.g., at least one treatment with another anticancer treatment, before the period of time.

In one embodiment, a method for treating biliary tract cancer comprises or consists essentially of administering to a patient in need thereof, during the period of time, a combination therapy comprising or consisting essentially of or consisting of therapeutically effective amounts, independently or in combination, of (a) a MEK inhibitor, which is binimetinib or a pharmaceutically acceptable salt thereof, wherein binimetinib is administered orally twice daily twice daily in the amount of about 10 mg to about 100 mg (e.g., any of the subranges or values in this range described herein, e.g., about 30 mg or about 45 mg), during the period of time and (b) a fluoropyrimidine-containing therapy which is capecitabine, wherein capecitabine is administered orally twice daily in the amount of about 1000 mg/m² or about 1250 mg/m² (e.g., any of the subranges or values in this range described herein) during the period of time. In one embodiment, binimetinib is administered twice daily for two weeks followed by a one week rest period for a three week period. In one embodiment, capecitabine is administered orally twice daily for two weeks followed by a one week rest period for a three week period. In one embodiment, binimetinib and capecitabine are each administered twice daily for two weeks followed by a one week rest period for a three week period, wherein the administration of binimetinib and capecitabine occur during the same two week period. In one embodiment, the patient was previously treated with one or more therapeutic agents, e.g., at least one treatment with another anticancer treatment, before the period of time. In one embodiment, the amounts of binimetinib and capecitabine together achieve a synergistic effect in the treatment of cancer (e.g., during the period of time). In one embodiment, the subject was previously treated with one or more prior therapies, e.g., one or more therapeutic agents, e.g., at least one treatment with another anticancer treatment, before the period of time.

In one embodiment, a method for treating biliary tract cancer comprises or consists essentially of administering to a patient in need thereof, during the period of time, a combination therapy comprising or consisting essentially of or consisting of therapeutically effective amounts, independently or in combination, of (a) a MEK inhibitor, which is binimetinib or a pharmaceutically acceptable salt thereof, wherein binimetinib is administered orally twice daily twice daily in the amount of about 15 mg during the period of time, and (b) a fluoropyrimidine-containing therapy which is capecitabine, wherein capecitabine is administered orally twice daily in the amount of about 1000 mg/m2. In one embodiment, binimetinib and capecitabine are administered for two weeks, followed by a one-week rest period during which binimetinib and capecitabine are not administered, for a three-week period. In one embodiment, the subject was previously treated with one or more prior therapies, e.g., one or more therapeutic agents, e.g., at least one treatment with another anticancer treatment, before the period of time.

In one embodiment, a method for treating biliary tract cancer comprises or consists essentially of administering to a patient in need thereof, during the period of time, a combination therapy comprising or consisting essentially of or consisting of therapeutically effective amounts, independently or in combination, of (a) a MEK inhibitor, which is binimetinib or a pharmaceutically acceptable salt thereof, wherein binimetinib is administered orally twice daily twice daily in the amount of about 30 mg during the period of time, and (b) a fluoropyrimidine-containing therapy which is capecitabine, wherein capecitabine is administered orally twice daily in the amount of about 1000 mg/m². In one embodiment, binimetinib and capecitabine are administered for two weeks, followed by a one-week rest period during which binimetinib and capecitabine are not administered, for a three-week period. In one embodiment, the subject was previously treated with one or more prior therapies, e.g., one or more therapeutic agents, e.g., at least one treatment with another anticancer treatment, before the period of time.

In one embodiment, a method for treating biliary tract cancer comprises or consists essentially of administering to a patient in need thereof, during the period of time, a combination therapy comprising or consisting essentially of or consisting of therapeutically effective amounts, independently or in combination, of (a) a MEK inhibitor, which is binimetinib or a pharmaceutically acceptable salt thereof, wherein binimetinib is administered orally twice daily twice daily in the amount of about 30 mg during the period of time, and (b) a fluoropyrimidine-containing therapy which is capecitabine, wherein capecitabine is administered orally twice daily in the amount of about 1250 mg/m². In one embodiment, binimetinib and capecitabine are administered for two weeks, followed by a one-week rest period during which binimetinib and capecitabine are not administered, for a three-week period. In one embodiment, the subject was previously treated with one or more prior therapies, e.g., one or more therapeutic agents, e.g., at least one treatment with another anticancer treatment, before the period of time.

In one embodiment, the invention is related to a method for treating biliary tract cancer comprising or consists essentially of administering to a patient in need thereof, during a period of time, a combination therapy comprising or consisting essentially of or consisting of an amount of a MEK inhibitor which is binimetinib or a pharmaceutically acceptable salt thereof and an amount of a fluoropyrimidine-containing therapy that is effective in treating biliary tract cancer. In another embodiment, the invention is related to a combination therapy method that consists essentially of administering to a patient in need thereof, over a period of time, a MEK inhibitor which is binimetinib or a pharmaceutically acceptable salt thereof, and a fluoropyrimidine-containing therapy. In another embodiment, the invention is related to a method for treating biliary tract cancer comprising or consisting essentially of administering to a patient in need thereof, over a period of time, a combination therapy comprising or consisting essentially of or consisting of an amount of a MEK inhibitor which is binimetinib or a pharmaceutically acceptable salt thereof, and an amount of a fluoropyrimidine-containing therapy, wherein the amounts together achieve synergistic effects in the treatment of cancer (e.g., during the period of time). In another embodiment, the invention is related to a combination therapy method consisting essentially of administering to a patient in need thereof, during a period of time, a MEK inhibitor which is binimetinib or a pharmaceutically acceptable salt thereof, and a fluoropyrimidine-containing therapy, wherein the amounts provide for a synergistic effect (e.g., in vivo or in vitro, e.g., in an appropriate model cell line or animal model). In one embodiment, the method or use of the invention is related to a synergistic combination therapy consisting essentially of a MEK inhibitor which is binimetinib or a pharmaceutically acceptable salt thereof, in combination with a fluoropyrimidine-containing therapy. In one aspect of all the embodiments of this paragraph, the fluoropyrimidine-containing therapy is capecitabine.

Those skilled in the art will be able to determine, according to known methods, the appropriate amount, dose or dosage of each compound, as used in the combination of the present invention, to administer to a patient, taking into account factors such as age, weight, general health, the compound administered, the route of administration, the nature and advancement of the cancer requiring treatment, and the presence of other medications.

The practice of the method of this invention may be accomplished through various administration or dosing regimens. The compounds of the combination of the present invention can be administered concurrently, sequentially, or intermittently, and in any order.

Repetition of the administration or dosing regimens may be conducted as necessary to achieve the desired effect. A “continuous dosing schedule”, as used herein, is an administration or dosing regimen without dose interruptions, e.g., without days off treatment. Repetition of 21 or 28 day treatment cycles without dose interruptions between the treatment cycles is an example of a continuous dosing schedule. In an embodiment, one or both components of the combination of the present invention can be administered in a continuous dosing schedule. An “intermittent dosing schedule” as used herein, is an administration or dosing regimen with dose interruptions. For example, in one embodiment an intermittent dosing schedule comprises administration of one or more components of a combination therapy for two weeks, followed by a one week rest period in a three-week cycle.

In one embodiment of any of the dosing regimens of a combination therapy as described herein, the second therapeutically effective dose of the MEK inhibitor is administered about 12 hours after the administration of the first dose of the MEK inhibitor, during the period of time. As used herein, the phrase “about 12 hours after the administration of the first dose of the MEK inhibitor” means that the second dose of the MEK inhibitor is administered 10 to 14 hours after the administration of the first dose of the MEK inhibitor, during the period of time.

In one embodiment of any of the dosing regimens of a combination therapy as described herein, the second therapeutically effective dose of the fluoropyrimidine-containing therapy is administered about 12 hours after the administration of the first dose of the fluoropyrimidine-containing therapy, during the period of time. As used herein, the phrase “about 12 hours after the administration of the first dose of the fluoropyrimidine-containing therapy” means that the second dose of the fluoropyrimidine-containing therapy is administered 10 to 14 hours after the administration of the first dose of the fluoropyrimidine-containing therapy, during the period of time.

In one embodiment, of any of the dosing regimens of a combination therapy as described herein, the first therapeutically effective dose of the fluoropyrimidine-containing therapy is administered at the same time as the administration of a therapeutically effective amount of the first therapeutically effective dose of the MEK inhibitor. In one embodiment, of any of the dosing regimens of a combination therapy as described herein, the second therapeutically effective dose of the fluoropyrimidine-containing therapy is administered at the same time as the administration of a therapeutically effective amount of the second therapeutically effective dose of the MEK inhibitor

In one embodiment, of any of the dosing regimens of a combination therapy as described herein, the first therapeutically effective dose of the fluoropyrimidine-containing therapy is administered at least 5 minutes after the administration of a therapeutically effective amount of the first therapeutically effective dose of the MEK inhibitor. As used herein, the phrase “at least 5 minutes after” means that the fluoropyrimidine-containing therapy is administered during the period of time at least 5 minutes, or at least 10 minutes, or at least 15 minutes, or at least 20 minutes, or at least 25 minutes, or at least 30 minutes, or at least 35 minutes, or at least 40 minutes, or at least 45 minutes, or at least 50 minutes, or at least 55 minutes, or at least 60 minutes, or at least 65 minutes, or at least 70 minutes, or at least 75 minutes, or at least 80 minutes, or at least 85 minutes, or at least 90 minutes after the administration of the first dose of the MEK inhibitor, during the period of time.

In one embodiment of any of the dosing regimens of a combination therapy as described herein, the first effective does of the therapeutically effective dose of the is administered at least 5 minutes before the administration of the first therapeutically effective dose of the MEK inhibitor, during the period of time. As used herein, the phrase “at least 5 minutes after” means that the fluoropyrimidine-containing therapy is administered during the period of time at least 5 minutes, or at least 10 minutes, or at least 15 minutes, or at least 20 minutes, or at least 25 minutes, or at least 30 minutes, or at least 35 minutes, or at least 40 minutes, or at least 45 minutes, or at least 50 minutes, or at least 55 minutes, or at least 60 minutes, or at least 65 minutes, or at least 70 minutes, or at least 75 minutes, or at least 80 minutes, or at least 85 minutes, or at least 90 minutes before administration of the first dose of the MEK inhibitor, during the period of time.

In one embodiment, the dose of the MEK inhibitor is escalated during the period of time until the Maximum Tolerated Dosage is reached, and the fluoropyrimidine-containing therapy is administered as a fixed dose, during the period of time. Alternatively, the MEK inhibitor may be administered as a fixed dose during the period of time and the dose of the fluoropyrimidine-containing therapy may be escalated until the Maximum Tolerated Dosage is reached, during the period of time.

In one embodiment, any combination therapy described herein may further comprise administration of one or more pre-medications prior to the administration of the fluoropyrimidine-containing therapy, during the period of time. In one embodiment, the one or more pre-medication(s) is administered during the period of time no sooner than 1 hour after administration of the MEK inhibitor. In one embodiment, the one or more premedication(s) is administered 30-60 minutes prior to the administration of the fluoropyrimidine-containing therapy, during the period of time. In one embodiment, the one or more premedication(s) is administered 30 minutes prior administration of the fluoropyrimidine-containing therapy, during the period of time. In one embodiment, the one or more pre-medications is selected from one or more of an anti-diarrheal, (e.g., loperamide), a H1 antagonist (e.g., antihistamines such as diphenhydramine) and acetaminophen.

In one embodiment, the one or more therapeutic agents that are administered to the patient before the period of time is or includes chemotherapy. In one embodiment, the one or more therapeutic agents that are administered to the patient before the period of time is or includes a platinum-based chemotherapy. In one embodiment, the one or more therapeutic agents that are administered to the patient before the period of time is or includes a fluoropyrimidine-containing chemotherapy (e.g., as a monotherapy). In one embodiment, the one or more therapeutic agents that are administered to the patient before the period of time is or includes gemcitabine. In one embodiment, the one or more therapeutic agents that are administered to the patient before the period of time is or includes FOLFIRINOX (a chemotherapy regimen of folinic acid (leucovorin), 5-FU (5-FU), irinotecan, and oxaliplatin). In one embodiment, the one or more therapeutic agents that are administered to the patient before the period of time is or includes FOLFOXIRI. In one embodiment, the one or more therapeutic agents that are administered to the patient before the period of time is or includes a targeted therapy.

An improvement in a cancer or cancer-related disease can be characterized as a complete or partial response. “Complete response” or “CR” refers to an absence of clinically detectable disease with normalization of any previously abnormal radiographic studies, bone marrow, and cerebrospinal fluid (CSF) or abnormal monoclonal protein measurements. “Partial response” refers to at least about a 10%, 20%, 30%, 40%, 50%, 60%, 70%, 80%, or 90% decrease in all measurable tumor burden (i.e., the number of malignant cells present in the subject, or the measured bulk of tumor masses or the quantity of abnormal monoclonal protein) in the absence of new lesions.

Treatment may be assessed with one or more clinical endpoints, for example by inhibition of disease progression, inhibition of tumor growth, reduction of primary tumor, relief of tumor-related symptoms, inhibition of tumor secreted factors (including expression levels of checkpoint proteins as identified herein), delayed appearance of primary or secondary tumors, slowed development of primary or secondary tumors, decreased occurrence of primary or secondary tumors, slowed or decreased severity of secondary effects of disease, arrested tumor growth and regression of tumors, increased Time To Progression (TTP), improved Time to tumor response (TTR), increased duration of response (DR), increased Progression Free Survival (PFS), increased Overall Survival (OS), Objective Response Rate (ORR), among others. OS as used herein means the time from treatment onset until death from any cause. TTP as used herein means the time from treatment onset until tumor progression; TTP does not comprise deaths. As used herein, TTR is defined for patients with confirmed objective response (CR or PR) as the time from the date of randomization or date of first dose of study treatment to the first documentation of objective tumor response. As used herein, DR means the time from documentation of tumor response to disease progression. As used herein, PFS means the time from treatment onset until tumor progression or death. As used herein, ORR means the proportion of patients with tumor size reduction of a predefined amount and for a minimum time period, where response duration usually is measured from the time of initial response until documented tumor progression. In the extreme, complete inhibition, is referred to herein as prevention or chemoprevention.

Thus, provided herein are methods for achieving one or more clinical endpoints associated with treating a cancer with a combination therapy described herein. In one embodiment, a patient described herein can show a positive tumor response, such as inhibition of tumor growth or a reduction in tumor size after treatment with a combination described herein. In certain embodiments, a patient described herein can achieve a Response Evaluation Criteria in Solid Tumors (for example, RECIST 1.1) of complete response, partial response or stable disease after administration of an effective amount a combination therapy described herein. In certain embodiments, a patient described herein can show increased survival without tumor progression. In some embodiments, a patient described herein can show inhibition of disease progression, inhibition of tumor growth, reduction of primary tumor, relief of tumor-related symptoms, inhibition of tumor secreted factors (including tumor secreted hormones, such as those that contribute to carcinoid syndrome), delayed appearance of primary or secondary tumors, slowed development of primary or secondary tumors, decreased occurrence of primary or secondary tumors, slowed or decreased severity of secondary effects of disease, arrested tumor growth and regression of tumors, decreased Time to Tumor Response (TTR), increased Duration of Response (DR), increased Progression Free Survival (PFS), increased Time To Progression (TTP), and/or increased Overall Survival (OS), among others.

In another embodiment, methods are provided for decreasing Time to Tumor Response (TTR), increasing Duration of Response (DR), increasing Progression Free Survival (PFS) of a patient having a cancer described herein, comprising administering an effective amount of a combination therapy as described herein. In one embodiment, a method is provided for decreasing Time to Tumor Response (TTR) of a patient having a cancer described herein, comprising administering an effective amount of a combination therapy as described herein. In one embodiment, is a method for increasing Progression Free Survival (PFS) of a patient a cancer described herein, comprising administering an effective amount of a combination therapy as described herein. In one embodiment, is a method for increasing Progression Free Survival (PFS) of a patient having a cancer described herein, comprising administering an effective amount of a combination therapy as described herein.

In one embodiment, the methods of treating biliary tract cancer according to the invention also include surgery or radiotherapy. Non-limiting examples of surgery include, e.g., open surgery or minimally invasive surgery. Surgery can include, e.g., removing an entire tumor, debulking of a tumor, or removing a tumor that is causing pain or pressure in the subject. Methods for performing open surgery and minimally invasive surgery on a subject having a cancer are known in the art. Non-limiting examples of radiation therapy include external radiation beam therapy (e.g., external beam therapy using kilovoltage X-rays or megavoltage X-rays) or internal radiation therapy. Internal radiation therapy (also called brachytherapy) can include the use of, e.g., low-dose internal radiation therapy or high-dose internal radiation therapy. Low-dose internal radiation therapy includes, e.g., inserting small radioactive pellets (also called seeds) into or proximal to a cancer tissue in the subject. High-dose internal radiation therapy includes, e.g., inserting a thin tube (e.g., a catheter) or an implant into or proximal to a cancer tissue in the subject, and delivering a high dose of radiation to the thin tube or implant using a radiation machine. Methods for performing radiation therapy on a subject having a cancer are known in the art.

It may be shown by established test models that a combination therapy described herein results in the beneficial effects described herein before. The person skilled in the art is fully enabled to select a relevant test model to prove such beneficial effects. The pharmacological activity of a combination therapy described herein may, for example, be demonstrated in an animal model and/or a clinical study or in a test procedure, for example as described below.

Suitable clinical studies are, for example, open label, dose escalation studies in patients with biliary tract cancer. Such studies may demonstrate in particular the synergism of the therapeutic agents of a combination therapy described herein. The beneficial effects on proliferative diseases may be determined directly through the results of these studies. Such studies may, in particular, be suitable for comparing the effects of a monotherapy using the MEK inhibitor and/or the fluoropyrimidine-containing therapy versus the effects of a combination therapy comprising the MEK inhibitor and the fluoropyrimidine-containing therapy.

The efficacy of the treatment may be determined in such studies, e.g., after 6, 12, 18 or 24 weeks by evaluation of symptom scores, e.g., every 6 weeks.

In some embodiments of any of the methods described herein, the patient is identified as having a tumor or a cancer cell having an upregulated level of MEK, a mutated MEK having increased activity as compared to a wildtype MEK, an upregulated level of a kinase upstream of MEK kinase (e.g., Ras (KRAS, HRAS, and/or NRAS) and/or Raf), or a mutated kinase upstream of MEK (e.g., Ras and/or Raf) having increased activity as compared to the corresponding wildtype kinase upstream of MEK.

In some embodiments, a mutated MEK having increased activity as compared to a wildtype MEK can have, e.g., one or more amino acid substitutions at an amino acid positions selected from the group of 56 (e.g., Q56P) and 72 (e.g., S72G).

In some embodiments, a mutated KRAS having increased activity as compared to a wildtype KRAS can have, e.g., one or more amino acid substitutions at amino acid position 12 (e.g., G12A, G12R, G12S, G12C, G12D or G12V), 13 (e.g., G13D or G13C).

In some embodiments, a mutated HRAS having increased activity as compared to a wildtype HRAS can have, e.g., one or both of amino acid substitutions at amino acid positions 12 (e.g., G12V) and 61 (e.g., Q61L or Q61R).

In some embodiments, a mutated NRAS having increased activity as compared to a wildtype NRAS can have, e.g., an amino acid substitution at one or more of amino acid positions 12 (e.g., G12D, G12S, or G12V), 13 (e.g., G13R or G13V), and 61 (e.g., Q61H, Q61K, Q61L, or Q61R).

In some embodiments, a mutated BRAF having increased activity as compared to a wildtype BRAF can have, e.g., an amino acid substitution at amino acid position 600 (e.g., V600E or V600K).

In some embodiments of any of the methods described herein, the patient is identified as having a tumor or a cancer cell having an upregulated level of EGFR (e.g., as compared to a non-cancerous cell) or a mutated EGFR having increased activity as compared to a wildtype EGFR.

In some embodiments, a mutated EGFR having increased activity as compared to a wildtype EGFR can have, e.g., an amino acid substitution at amino acid position 719, an amino acid substitution at amino acid position 731 (e.g., W731L), an amino acid substitution at amino acid position 734 (e.g., E734Q), an amino acid substitution at amino acid position 785 (e.g., T785A), an amino acid substitution at amino acid position 790 (e.g., T790M), an amino acid substitution at amino acid position 797 (e.g., C797Y), an amino acid substitution at amino acid position 801 (e.g., Y801H), an amino acid substitution at amino acid position 831 (e.g., L831H), an amino acid substitution at amino acid position 858 (e.g. L858R), an amino acid substitution at amino acid position 861 (e.g., L861Q), and an amino acid substitution at amino acid position 868 (e.g., E868G).

Methods for detecting an increased level of MEK, Ras, Raf, and/or EGFR or expression of a mutated MEK, Ras, Raf, and/or EGFR that has increased activity as compared to the corresponding wildtype kinase in a tumor (e.g., a biopsy sample) or a cancer cell are known in the art and include, e.g., nucleic acid sequencing (e.g., PCR), fluorescence in situ hybridization (FISH) with a labeled DNA probe, immunofluorescence microscopy, immunoblotting, proteomics, mass spectrometry, and fluorescence-assisted cell sorting. Additional methods for detecting an increased level of MEK, Ras, Raf, and/or EGFR, or expression of a mutated MEK, Ras, Raf, and/or EGFR that has increased activity as compared to the corresponding wildtype kinase in a tumor (e.g., a biopsy sample) or a cancer cell are known in the art.

Some embodiments of any of the methods described herein further include identifying a patient as having a tumor or a cancer cell that has an increased level of MEK, Ras, Raf, and/or EGFR, or expresses a mutated MEK, Ras, Raf, and/or EGFR that has increased activity as compared to the corresponding wildtype kinase, and selecting the identified patient for treatment using any of the methods described herein. Some embodiments of any of the methods described herein can further include a step of selecting a subject identified as having a tumor or a cancer cell that has an increased level of MEK, Ras, Raf, and/or EGFR, or expresses a mutated MEK, Ras, Raf, and/or EGFR that has increased activity as compared to the corresponding wildtype kinase, and the treating the patient using any of the methods described herein.

In some embodiments, the cancer is selected from the group consisting of: pancreatic cancer, breast cancer (e.g., triple-negative breast cancer), mantle cell lymphoma, non-small cell lung cancer, melanoma, colon cancer, esophageal cancer, liposarcoma, multiple myeloma, T-cell leukemia, renal cell carcinoma, gastric cancer, glioblastoma, hepatocellular carcinoma, lung cancer, colorectal cancer, rhabdoid tumor, retinoblastoma proteinpositive cancers, gallbladder cancer, cholangiocarcinoma (e.g., intrahepatic cholangiocarcinoma and extrahepatic cholangiocarcinoma), ampulla of Vater cancer, astrocytomas, glioblastoma multiforme, Bannayan-Zonana syndrome, Cowden disease, Lhermitte-Duclos disease, Wilm's tumor, Ewing's sarcoma, Rhabdomyosarcoma, ependymoma, medulloblastoma, head and neck, kidney cancer, ovarian cancer, prostate cancer, sarcoma, osteosarcoma, giant cell tumor of bone, thyroid, Chronic myelogenous leukemia, Chronic lymphocytic leukemia, Hairy-cell leukemia, acute lymphoblastic leukemia, acute myelogenous leukemia, AML, Chronic neutrophilic leukemia, plasmacytoma, Immunoblastic large cell leukemia, Mantle cell leukemia, Megakaryoblastic leukemia, acute megakaryocytic leukemia, promyelocytic leukemia, Erythroleukemia, malignant lymphoma, Hodgkins lymphoma, non-Hodgkins lymphoma, lymphoblastic T cell lymphoma, Burkitt's lymphoma, follicular lymphoma, neuroblastoma, bladder cancer, urothelial cancer, lung cancer, vulval cancer, cervical cancer, endometrial cancer, renal cancer, mesothelioma, salivary gland cancer, nasopharangeal cancer, buccal cancer, cancer of the mouth, GIST (gastrointestinal stromal tumor), NSCLC, and testicular cancer. In some embodiments, the cancer is a T-cell infiltrating cancer.

In some embodiments, the cancer is selected from the group consisting of: non-small cell lung cancer, biliary tract cancer, breast cancer, bladder cancer, cervical cancer, malignant mesothelioma, ovarian cancer and pancreatic cancer.

The compounds of the method or combination of the present invention may be formulated prior to administration. The formulation will preferably be adapted to the particular mode of administration. These compounds may be formulated with pharmaceutically acceptable carriers as known in the art and administered in a wide variety of dosage forms as known in the art. In making the pharmaceutical compositions of the present invention, the active ingredient will usually be mixed with a pharmaceutically acceptable carrier, or diluted by a carrier or enclosed within a carrier. Such carriers include, but are not limited to, solid diluents or fillers, excipients, sterile aqueous media and various non-toxic organic solvents. Dosage unit forms or pharmaceutical compositions include tablets, capsules, such as gelatin capsules, pills, powders, granules, aqueous and nonaqueous oral solutions and suspensions, lozenges, troches, hard candies, sprays, creams, salves, suppositories, jellies, gels, pastes, lotions, ointments, injectable solutions, elixirs, syrups, and parenteral solutions packaged in containers adapted for subdivision into individual doses.

Parenteral formulations include pharmaceutically acceptable aqueous or nonaqueous solutions, dispersion, suspensions, emulsions, and sterile powders for the preparation thereof. Examples of carriers include water, ethanol, polyols (propylene glycol, polyethylene glycol), vegetable oils, and injectable organic esters such as ethyl oleate. Fluidity can be maintained by the use of a coating such as lecithin, a surfactant, or maintaining appropriate particle size. Exemplary parenteral administration forms include solutions or suspensions of the compounds of the invention in sterile aqueous solutions, for example, aqueous propylene glycol or dextrose solutions. Such dosage forms can be suitably buffered, if desired.

Additionally, lubricating agents such as magnesium stearate, sodium lauryl sulfate and talc are often useful for tableting purposes. Solid compositions of a similar type may also be employed in soft and hard filled gelatin capsules. Preferred materials, therefor, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art.

In one embodiment, the MEK inhibitor which is binimetinib or a pharmaceutically acceptable salt thereof is formulated for oral administration. In one embodiment, the MEK inhibitor is formulated as a tablet or capsule. In one embodiment, the MEK inhibitor is formulated as a tablet. In one embodiment, the tablet is a coated tablet. In one embodiment, the MEK inhibitor is binimetinib as the fee base. In one embodiment, the MEK inhibitor is a pharmaceutically acceptable salt of binimetinib. In one embodiment, the MEK inhibitor is crystallized binimetinib. Methods of preparing oral formulations of binimetinib are described in PCT publication No. WO 2014/063024. In one embodiment, a tablet formulation of binimetinib comprises 15 mg of binimetinib. In one embodiment, a tablet formulation of binimetinib comprises 15 mg of crystallized binimetinib. In one embodiment, a tablet formulation of binimetinib comprises 45 mg of binimetinib. In one embodiment, a tablet formulation of binimetinib comprises 45 mg of crystallized binimetinib.

The invention also relates to a kit comprising the therapeutic agents of the combination of the present invention and written instructions for administration of the therapeutic agents. In one embodiment, the written instructions elaborate and qualify the modes of administration of the therapeutic agents, for example, for simultaneous or sequential administration of the therapeutic agents of the present invention. In one embodiment, the written instructions elaborate and qualify the modes of administration of the therapeutic agents, for example, by specifying the days of administration for each of the therapeutic agents during a 28 day cycle.

Although the disclosed teachings have been described with reference to various applications, methods, kits, and compositions, it will be appreciated that various changes and modifications can be made without departing from the teachings herein and the claimed invention below. The foregoing examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

### EXAMPLES

### Example 1

### Preclinical Study

The effect of binimetinib, 5-fluorouracil (5-FU), or combination of these drugs on cell viability was evaluated using eight BTC cell lines (SNU245, SNU308, SNU478, SNU869, SNU1079, SNU1196, TFK1, and HuCCT1). As capecitabine is converted to 5-FU in vivo, 5-FU was used as an in vivo equivalent of oral administration of capecitabine in this preclinical study in cell lines. In brief, confluent monolayers, which had been grown in 96 well plates, were exposed to drugs for 72 hours. Subsequently, cell viability was measured at 540 nm with a Multiskan GO microplate reader using a commercially available MTT assay, according to the manufacturer's directions. The Chou-Talalay method was used to assess a combination effect. Cells were also treated with drugs for 48 hours and the expression of thymidylate synthase (TS), programmed death-ligand 1 (PD-L1), and β-actin was determined using western blot analysis according to a published protocol. Actin was included as a loading control.

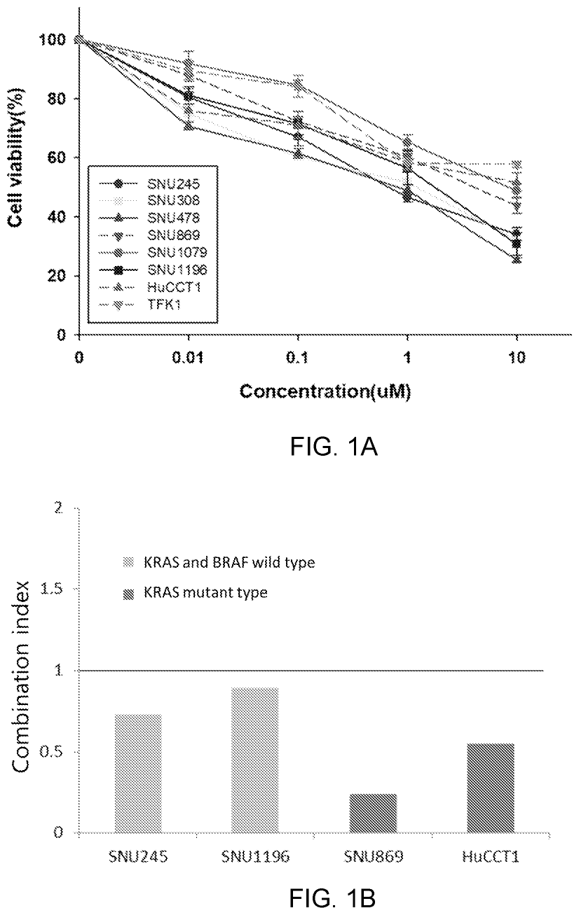

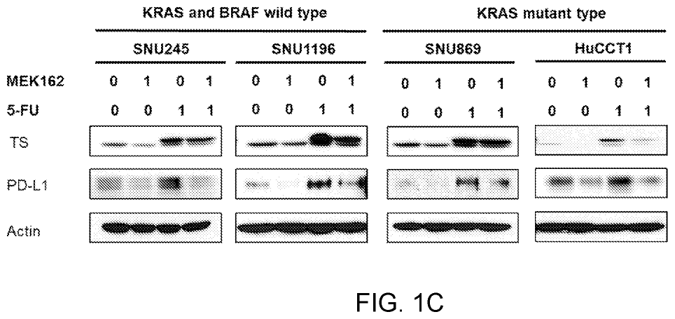

Exposure of the BTC cell lines to binimetinib was associated with significant decreases in cell viability (FIG. 1A). The combination of binimetinib and 5-FU in SNU245, SNU1196, SNU869, and HuCCT1 demonstrated synergistic effects (combination index <1 at fraction affected=0.5; FIG. 1B). Of these cell lines, SNU869 and HuCCT1 have the KRAS mutation (G12D). To evaluate the underlying synergistic mechanism, protein expression was evaluated by western blot. Thymidylate synthase (TS), a marker of 5-FU resistance, was upregulated by 5-FU treatment. However, TS was downregulated by binimetinib monotherapy (FIG. 1C). When the BTC cells were treated with both binimetinib and 5-FU, the TS levels induced by 5-FU were also downregulated by addition of binimetinib, which may increase the sensitivity of 5-FU. Interestingly, 5-FU induced PD-L1 expression, and co-administration with binimetinib and 5-FU decreased this expression (FIG. 1C).

### Example 2

### Phase Ib Study of Binimetinib (MEK162) in Combination with Capecitabine in Gemcitabine-Pretreated Advanced Biliary Tract Cancer

In biliary tract cancer (BTC), the RAS/RAF/MEK/ERK pathway is known have increased activity (e.g., as compared to a non-cancerous cell) in up to 20-40% of cases. Binimetinib (MEK162) is an allosteric MEK1/2 inhibitor, which shows preclinical activity in BTC. MEK inhibitor and 5-FU showed synergistic effects in BTC cells.

Supported by the preclinical results (Example 1), a phase Ib study of binimetinib and capecitabine in gemcitabine-pretreated BTC patients was conducted to assess the safety and early antitumor activity. Furthermore, genetic alterations to the RAS/RAF/MEK/ERK pathway were identified and plasma biomarker concentrations were determined.

Study Design

A phase Ib study using binimetinib and capecitabine was conducted in advanced BTC patients who were previously treated with gemcitabine-based chemotherapy. This study consisted of a dose escalation (DE) part and an expansion part (EX). The primary endpoint of the DE part was determination of the MTD, and secondary endpoints included identification of dose-limiting toxicity (DLT), the RP2D, and safety. Binimetinib (B) and capecitabine (C) were dosed twice daily, 2 weeks on/1 week off, every 3 weeks. In the dose escalation part, 3 dose levels (DL) were tested (DL1: 1000 mg/m² of C and 15 mg of B; DL2: 1000 mg/m² of C and 30 mg of B; DL3: C 1250 mg/m² and 30 mg of B) according to a “3+3 design”. The primary end-point of the EX part of the study was to determine the 3-month progression-free survival (PFS) rate and secondary endpoints were the objective response rate (ORR), response duration, disease control rate (DCR), PFS, overall survival (OS), safety, quality of life (QOL), and biomarker quantitation.

Patients

For the dose escalation part of the study, 9 patients (3 per DL) were recruited. The target population of this study was BTC patients in their second- or third-line treatment setting who had failed a gemcitabine-based first-line chemotherapy. The major inclusion criteria were aged ≥20 years; histologically confirmed BTC including, intrahepatic cholangiocarcinoma, extrahepatic cholangiocarcinoma, gallbladder cancer, or ampulla of Vater cancer; unresectable or recurrent disease; prior gemcitabine-based chemotherapy; Eastern Cooperative Oncology Group (ECOG) performance status 0-1; evaluable or measurable lesions by Response Evaluation Criteria In Solid Tumors version 1.1 (RECIST v1.1); adequate bone marrow and organ function; and corrected QT interval ≤480 ms. The major exclusion criteria were active central nervous system disease; brain metastasis; risk or history of retinal vein occlusion; transplantation history; Gilbert syndrome; major heart disease within 6 months; neuromuscular disease related with elevation of creatine kinase. Biliary drainage was allowed. All participating patients provided a written informed consent. This study was approved by the institutional review boards of each hospital and was registered with the ClinicalTrials.gov (Identifier: NCT02773459).

Dosing and Dose Modification

Binimetinib and capecitabine were orally administered twice daily, on days 1-14, every 3 weeks. In the dose-escalation part, 4 predefined dose levels (DLs) were applied (DL-1: binimetinib/capecitabine, 15 mg/800 mg/m²; DL1: 15 mg/1000 mg/m²; DL2: 30 mg/1000 mg/m²; DL3: 30 mg/1250 mg/m²). The starting dose was DLL If no patient experienced DLT in DL3, then DL3 was to be declared as the RP2D. DLT was predefined as grade 4 neutropenia with fever and/or infection; grade 4 neutropenia for ≥7 days; grade 3/4 thrombocytopenia with hemorrhage or transfusion; grade 4 thrombocytopenia for ≥7 days; grade 3 or 4 non-hematologic adverse events except alopecia, anorexia, nausea, or vomiting; or grade 3 or 4 nausea, diarrhea, or vomiting despite maximum supportive care. Dose reductions of binimetinib to 15 mg twice daily and capecitabine to 75 or 50% of the dose were permitted based on the protocol-defined treatment modifications.

Assessment of Response, Adverse Events, and QOL

Radiologic assessment was completed by computed tomography (CT) every 6 weeks, and tumor response was evaluated based on RECIST v1.1. Routine evaluation including physical examinations and vital signs, and assessment of adverse events were completed weekly during the first cycle, and then at the end of every cycle. Adverse events were recorded using the NCI—Common Terminology Criteria of Adverse Events version 4.03. QOL was evaluated through the use of the EORTC-QLQ-C30 and EQ5D questionnaires (see Aaronson N K, et al. J Natl Cancer Inst 85:365-76, 1993 and Health Policy 16:199-208, 1990; the disclosures of which are incorporated herein by reference in their entireties), which were collected at baseline, after cycles 1, 2, 4, 6, and 8, and then after every third cycle until the end of study.

Biomarker Analysis

All patients were required to provide tumor tissues at screening, and blood samples at screening, after the first cycle, after second cycle, and at disease progression. Genetic alteration was assessed using targeted sequencing by next-generation sequencing (NGS) to determine mutations of RAS/RAF/MEK/ERK pathway. To predict treatment efficacy, interleukin-6 (IL-6) plasma concentrations were evaluated. The plasma concentrations of IL-6 were measured using an enzyme-linked immunosorbent assay (Human IL-6, Quantikine ELISA Kit, R&D systems, Minneapolis, Minn., USA) according to the manufacturer's instructions. Each sample was analyzed in duplicate.

Statistical Analysis

The safety and efficacy analysis were completed in an intent-to-treat population who received at least one dose of binimetinib. The continuous variables were summarized using descriptive statistics, such as the mean and standard deviation, or median and range, whereas categorical variables were summarized using frequencies and percentages. Cutpoint values of each continuous variable for OS and PFS predictions were determined by finding the optimal cutpoint for continuous covariates with time-to-event outcomes (see Williams B A: Finding optimal cutpoints for continuous covariates with binary and time-to-event outcomes. Technical Report Series #79, June 2006, Department of Health Sciences Research, Mayo Clinic, Rochester Minn.). The results of the EORTC-QLQ-C30 were interpreted in line with the method of Osoba et al (see Osoba D, et al., J Clin Oncol 16:139-44, 1998). The PFS was calculated from the date of first cycle to the development of progressive disease (PD) or death, regardless of cause. The OS was calculated from the date of first cycle to death, regardless of the cause. All analyses were performed using PASW Statistics 18 (SPSS Inc., Chicago, Ill.) and R Statistical Software (R version 3.4.4).

Results

Patients

Nine patients were recruited for the dose-escalation part. None of the patients experienced DLT up to DL3, therefore the RP2D was determined as DL3 (binimetinib 30 mg/capecitabine 1250 mg/m², twice daily on days 1-14, every 3 weeks). For the expansion part, 25 patients were enrolled. At the data cut-off time, 22 patients had died, and treatment was ongoing in 3 patients. The median follow-up duration was 6.8 months (range, 2.0-17.8) and patients received a median of 5 cycles (range, 1-16).

Baseline patient characteristics were not significantly different between the dose-escalation and expansion part. The median age was 63 years old (range, 48-73). The Primary tumor origins were the gallbladder (n=10, 29.4%), intrahepatic bile duct (n=10, 29.4%), extrahepatic bile duct (n=9, 26.5%), and ampulla of Vater (n=5, 14.7%). Twenty-five (73.5%) patients were in their second-line treatment setting and 9 patients (26.5%) were in their third-line setting. Twelve patients (35.3%) had previously been exposed to 5-FU as an adjuvant or during a palliative chemotherapy period, and among them, 4 patients (33.3%) had experienced 5-FU failure as first- or second-line treatment.

Adverse Events

The majority of the adverse events were both manageable and reversible. Of note, during the first cycle in dose-escalation part, one G3 neutropenia and one G3 thrombocytopenia occurred, but there was no DLT. The most common adverse events were stomatitis (61.7%), edema (50.0%), nausea (41.2%), papulopustular rash (41.2%), palmar-plantar erythrodysesthesia syndrome (41.2%), and fatigue (41.2%). There were no ocular adverse events and only one patient experienced grade 4 toxicity (hypokalemia). There were no treatment-related deaths.

Treatment Efficacy

All patients had at least one measurable lesion. Of 34 patients, 7 patients (20.6%) and 19 patients (55.9%) demonstrated partial response (PR) and stable disease (SD), respectively (Table 1).

TABLE 1
Tumor response, progression-free survival, and overall survival.

| | Total | Second-line | Third-line | P |
|---|---|---|---|---|
| | (n = 34) | (n = 25) | (n = 9) | value* |
| Response | | | | |
| Complete | 0 (0.0%) | 0 (0.0%) | 0 (0.0%) | 0.988 |
| response | | | | |
| Partial | 7 (20.6%) | 5 (20.0%) | 2 (22.2%) | |
| response | | | | |
| Stable disease | 19 (55.9%) | 14 (56.0%) | 5 (55.6%) | |
| Progressive | 8 (23.5%) | 6 (24.0%) | 2 (22.2%) | |
| disease | | | | |
| Objective | 20.6% | 20.0% | 22.2% | |
| response rate | | | | |
| Disease | 76.5% | 76.0% | 77.8% | |
| control rate | | | | |
| Progression- | 4.1 months | 4.4 months | 3.5 months | 0.064 |
| free survival | (95% CI, | (95% CI, | (95% CI, | |
| | 2.8-5.7) | 2.9-5.9) | 2.2-4.8) | |
| Overall | 7.8 months | 7.8 months | 6.8 months | 0.796 |
| survival | (95% CI, | (95% CI, | (95% CI, | |
| | 5.9-12.2) | 5.4-10.2) | 5.6-8.0) | |

*the comparison between second-line and third-line.
CI, confidence interval

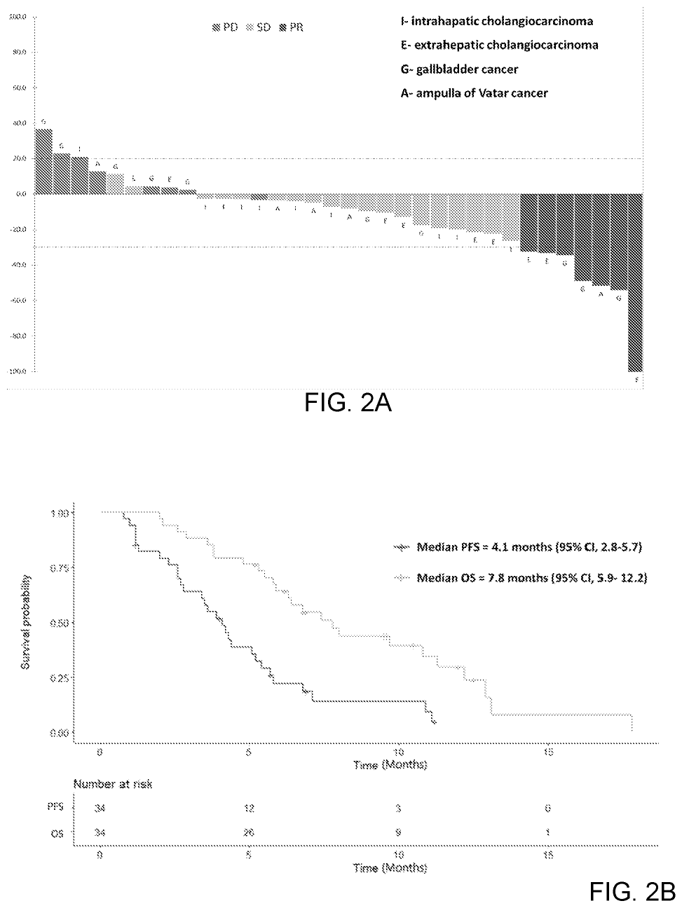

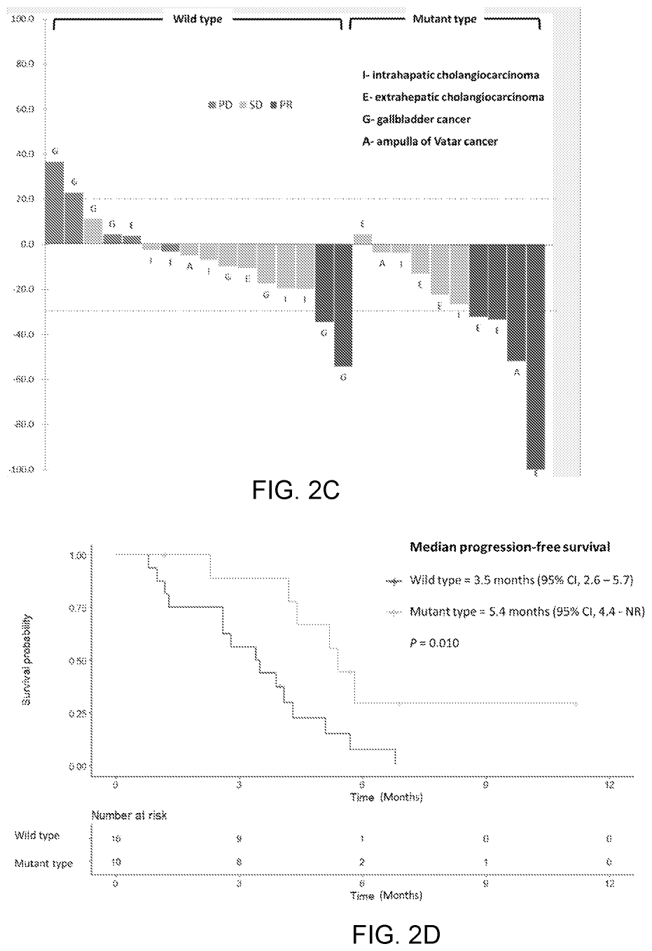

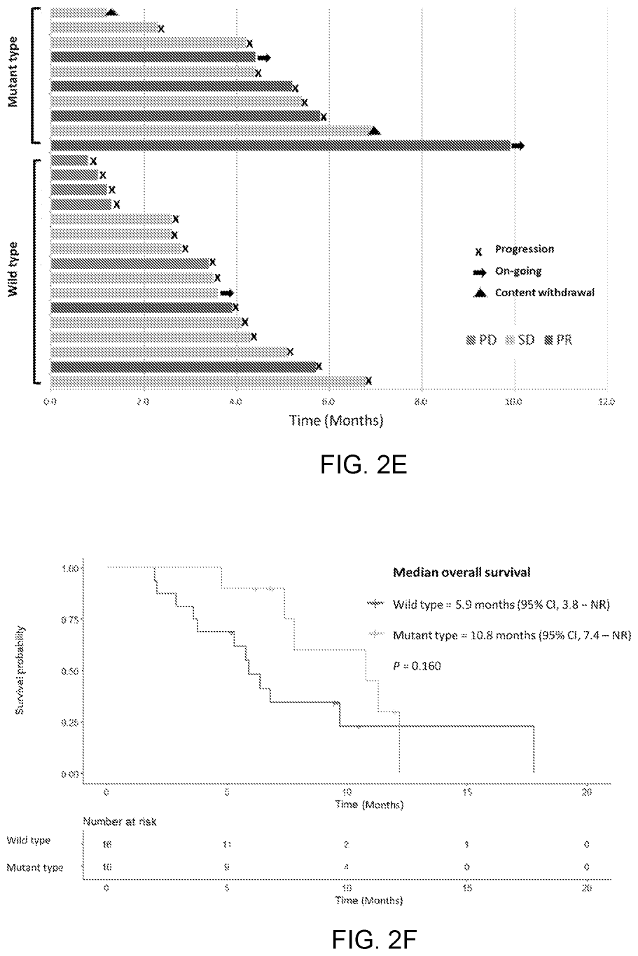

The ORR and DCR were 20.6% (95% confidence interval (CI), 7.0-34.2) and 76.5% (95% CI, 62.2-90.8), respectively. Twenty-five patients (73.5%) experienced tumor shrinkage with any grade (FIG. 2A) and the median response duration was 4.7 months (95% CI, 2.4-7.0). Of the 19 patients with SD, 13 (68.4%) demonstrated durable disease control with SD duration for greater than 12 weeks. The tumor response was similar between second- and third-line settings (Table 1). Tumor origin also did not alter tumor response. Furthermore, of the 4 patients who had failed 5-FU at the first- or second-line setting, one patient (25%) showed PR and the other 3 patients (75%) showed SD.

In all patients, the 3-month PFS rate was 64.0% and the median PFS was 4.1 months (95% CI, 2.8-5.7; FIG. 2B). The median OS was 7.8 months (95% CI, 5.9-12.2). Neither the PFS nor OS were significantly different between second- and third-line settings (p=0.064, p=0.796, respectively). There was also no significant difference in the PFS or OS when patients were grouped according to tumor origin (p=0.158, p=0.091, respectively).

Biomarker Analysis

In all patients, tumor tissues were obtained during screening. However, genomic sequence information using NGS techniques was obtained for 26 of the 34 participants (76.5%). Genetic alterations in the RAS/RAF/MEK/ERK pathway were identified in 10 (38.5%) of 26 patients (Table 2).

TABLE 2
Mutations in the RAS/RAF/MEK/ERK pathway

| | Mutation site |
|---|---|
| Patient 1 | KRAS (G12D) |
| Patient 2 | MAP2K1 (E203K) |
| Patient 3 | KRAS (G12A) |
| Patient 4 | KRAS (G12C) |
| Patient 5 | NRAS (Q61L) |
| Patient 6 | KRAS (G12V) |
| Patient 7 | KRAS (G12V) |
| Patient 8 | KRAS (G12V) |
| Patient 9 | KRAS (G12D) |
| Patient 10 | MAP2K1 (E203V) |

Furthermore, patients with mutations in the RAS/RAF/MEK/ERK pathway responded significantly better to therapy than those with wild-type (40.0% vs 12.5%; FIG. 2C and Table 3).

TABLE 3
Tumor response, progression-free survival, and
overall survival according to mutation status
within the RAS/RAF/MEK/ERK pathway
(online only)

| | Mutant type | Wild type | |
|---|---|---|---|
| Response | (n = 10) | (n = 16) | P value |
| Complete response | 0 (0.0%) | 0 (0.0%) | 0.028 |
| Partial response | 4 (40.0%) | 2 (12.5%) | |
| Stable disease | 6 (60.0%) | 9 (56.2%) | |
| Progressive disease | 0 (0.0%) | 5 (31.2%) | |
| Objective response | 40.0% | 12.5% | |
| rate | | | |
| Disease control rate | 100.0% | 68.8% | |
| Progression-free | 5.4 months | 3.5 months | 0.010 |
| survival | (95% CI, | (95% CI, | |
| | 4.4-NR) | 2.6-5.7) | |
| Overall survival | 10.8 months | 5.9 months | 0.160 |
| | (95% CI, | (95% CI, | |
| | 7.4-NR) | 3.8-NR) | |

CI, confidence interval;
NR, not reached.

Patients with mutant-type also showed longer PFS (5.4 months vs 3.5 months; FIG. 2D and FIG. 2E) and OS (10.8 months vs 5.9 months; FIG. 2F) than those with wild-type.

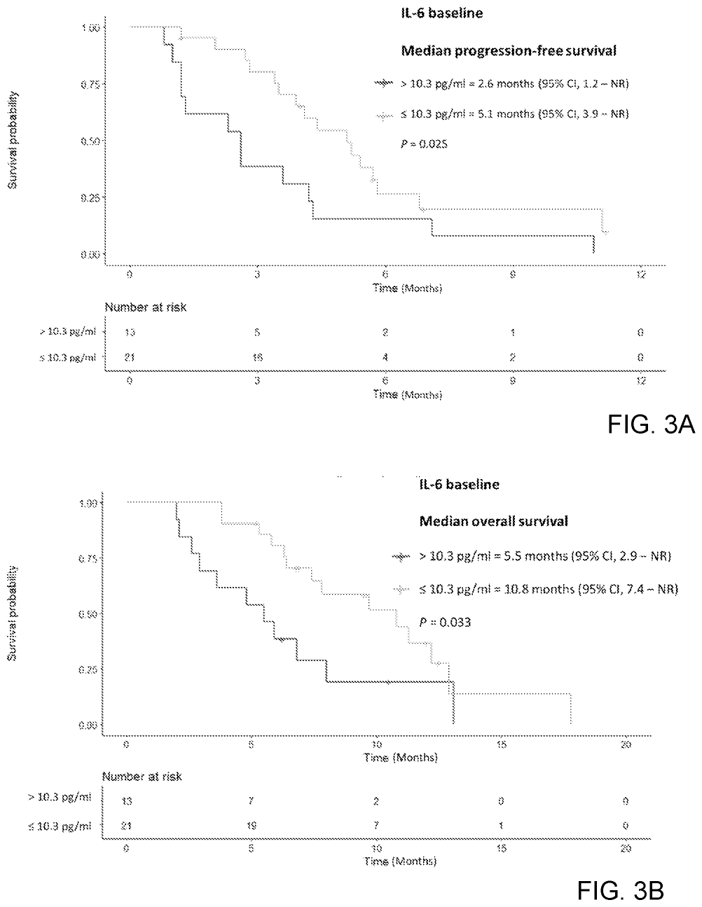

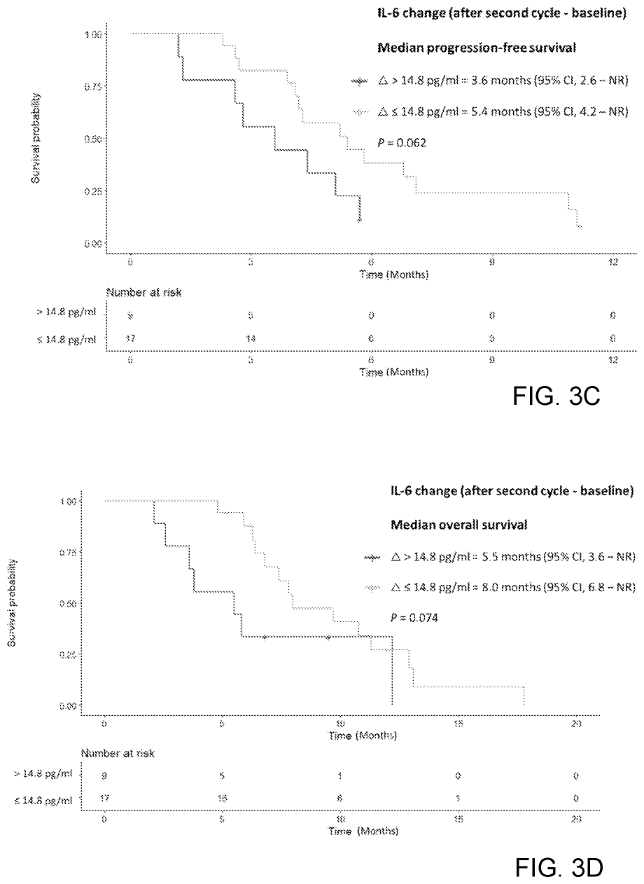

In terms of IL-6 plasma concentrations, the mean value (±standard deviation) of baseline plasma IL-6 was 11.5 pg/mL (±12.6). Patients with higher baseline IL-6 showed significantly shorter PFS and OS (p=0.025, p=0.033, respectively; FIGS. 3A and 3B). Similarly, the baseline value of IL-6 was associated with tumor response, that is, IL-6 was higher in PD than PR patients. The mean concentrations were 19.9, 9.2, and 7.7 pg/mL for PD, SD, and PR patients, respectively (p=0.085). With regard to changes between baseline and after the second cycle, a greater increase in the IL-6 concentration (Δ>14.8 pg/mL) was associated with shorter PFS and OS (FIGS. 3C and 3D). Furthermore, the plasma concentrations of IL-6 when PD was confirmed were also significantly increased relative to baseline (mean±standard deviation: 32.0±29.8 vs 9.0±5.9 pg/mL, respectively; paired t-test, p=0.008).

QOL

Based on the EORTC-QLQ-C30 questionnaire, most QOLs regarding global health status and functioning were altered between the degrees of ‘a little’ to ‘very much’ as the cycles proceeded. QOLs related to symptoms demonstrably improved at some time points. Compared best status to baseline, role functioning and financial difficulties improved with ‘a little’ degree (p=0.028 and p=0.032, respectively), and QOL related to pain improved with ‘moderate’ degree (p=0.039). The EQ5D questionnaire also demonstrated alterations of the QOL throughout the treatment period.

Summary of the Results

In the DE part, 9 patients were recruited and no dose limiting toxicity was noted. Therefore, the recommended phase 2 dose was determined as DL3. In EX part, 25 patients were enrolled. Of the total 34 patients, 25 (73.5%) and 9 patients (26.5%) were second-line and third-line setting, respectively. The 3-month PFS rate was 64.0%, and the median PFS and overall survival (OS) were 4.1 (95% CI, 2.8-5.7) and 7.8 months (95% CI, 5.9-12.2), respectively. The objective response rate and disease control rate were 20.6% (95% CI, 7.0-34.2) and 76.5% (95% CI, 62.2-90.8), respectively. 68.4% of stable diseases were durable (>12 weeks). Furthermore, patients with RAS/RAF/MEK/ERK pathway mutations (38.5%) showed significantly better tumor response (p=0.028), PFS (5.4 vs 3.5 months, p=0.010) and OS (10.8 vs 5.9 months, p=0.160) than wild-type. Most of adverse events were grade ½ and manageable.

Six patients (17.6%) and 20 patients (58.8%) showed partial response and stable disease (SD), respectively. Response rate and disease control rates were 17.6% (95% CI, 4.8-30.4) and 76.5% (95% CI, 62.1-90.7), respectively. Median PFS was 3.9 months (95% CI, 3.0-4.8) and median overall survival (OS) was 8.0 months (95% CI, 4.9-11.1). Three-month progression free survival (3m-PFSR) was 61.3%. Sixty percent of patients with SD showed prolonged SD (>12 weeks). In a biomarker study, RAS/RAF/MEK/ERK pathway activated patients showed longer PFS (5.4 m vs 2.6 m, p=0.031) and OS (10.8 vs 5.3 m, p=0.011) than non-activated patients.

The RP2D of binimetinib and capecitabine combination is 1250 mg/m² of capecitabine and 30 mg of binimetinib, twice daily, 2 weeks on/1 week off. This combination showed acceptable tolerability and promising antitumor efficacy, especially in BTC patients having increased RAS/RAF/MEK/ERK signaling activity.

### DISCUSSION

This study is the first proof-of-concept trial to evaluate the safety and efficacy of binimetinib in combination with capecitabine for gemcitabine-pretreated BTC, and was supported by preclinical data suggestive of synergism between binimetinib with fluoropyrimidine. This combination demonstrated promising antitumor efficacy, especially in BTC patients with mutations in the RAS/RAF/MEK/ERK pathway.

A previous Phase Ib study of binimetinib monotherapy in advanced or metastatic BTC patients reported an ORR of 8% and a DCR of 51%, as well as PFS of 2.1 months and OS of 4.8 months, respectively (Finn R S, et al.: Phase 1b investigation of the MEK inhibitor binimetinib in patients with advanced or metastatic biliary tract cancer. Invest New Drugs, May 22, 2018. In contrast, the ORR, DCR, PFS, and OS were 20.6%, 76.5%, 4.1 months, and 7.8 months, in the present study. Considering that our study consisted of patients in their second-(73.5%) and third-line settings (26.5%), the efficacy of the present study may be better than the aforementioned binimetinib monotherapy study, in which more than half the patient populations were chemotherapy-naïve in metastatic setting (57%). Furthermore, a systematic review of patients in the second- and third-line setting for BTC described ORR and DCR of less than 10% and less than 50%; the PFS and OS were around 3 months and 6 months (Lamarca A, et al., Ann Oncol 25:2328-38, 2014). Moreover, a previous study, which included both second- and third-line setting BTC patients treated with a 5-FU based combination treatment (infusional FAM regimen), had a similar population to the present study, and reported PFS and OS values of 2.4 months and 6.1 months, respectively (Lim K H, et al., Cancer Chemother Pharmacol 68:1017-26, 2011). Considering these results, the efficacy of binimetinib and capecitabine in the present study is very encouraging and is likely attributed to the synergism between binimetinib and capecitabine. Indeed, preclinical experiments demonstrated the downregulation of TS and PD-L1 induced by 5-FU in response to binimetinib. Interestingly, in the present study, all patients who had previously failed 5-FU-based chemotherapy achieved either a partial response (PR) or stable disease (SD) with the combination of binimetinib and capecitabine, which is likely attributed to synergism.

Activation of the MEK pathway, such as through RAS or BRAF mutations, has been reported as a predictive marker for the success of MEK inhibitors (Horiuchi H, et al., Int J Oncol 23:957-63, 2003; Solit D B, et al., Nature 439:358-62, 2006). In the present study, genetic mutations within the RAS/RAF/MEK/ERK pathway were identified in 10 (38.5%) out of 26 patients whose NGS data were obtained. This incidence was in accordance with previous studies. Importantly, this study demonstrates that a tissue-based biomarker selection strategy for BTC patient management or enrolling BTC patients into clinical trials is doable and feasible. Interestingly, patients with mutations in the RAS/RAF/MEK/ERK pathway were associated with a higher ORR and longer survival than those with wild-type. Considering the patients in the present study were in the second- or third-line setting of BTC, these results of ORR (40%), PFS (5.4 months), and OS (10.8 months) were very promising in patients with mutant type of RAS/RAF/MEK/ERK pathways. Therefore, mutations in the RAS/RAF/MEK/ERK pathway could be a predictive biomarker for effective binimetinib treatment of BTC.

Immune modulation is another antitumor mechanism of MEK inhibitors, as MEK inhibition was reported to reduce the secretion of IL-6, which is associated with BTC tumor growth (Tai Y T, et al., Blood 110:1656-63, 2007; Park J, Tadlock L, et al., Hepatology 30:1128-33, 1999; Meng F, et al., J Hepatol 44:1055-65, 2006; Wehbe H, et al., Cancer Res 66:10517-24, 2006). In the present study, higher baseline concentrations of IL-6 were associated with worse prognosis. Furthermore, after the second cycle of treatment, patients with a larger increase were associated with worse prognosis. Therefore, early comparative determination of IL-6 between baseline and after treatment may predict disease outcomes in the binimetinib treatment.

In the present study, predefined 4 DLs were tested, and no DLT was observed. The highest DL (binimetinib 30 mg, capecitabine 1250 mg/m², twice daily on days 1-14, every 3 weeks) was determined as the RP2D. This dosage of binimetinib was relatively low in comparison with the monotherapy study, and a 1 week drug holiday every 3 weeks was introduced. This combination dosing schedule, which allowed full-dose capecitabine, was tolerable and adverse events were manageable. There were also no reports of ocular toxicity in this study. The drug combination was well tolerated, associated with manageable adverse events, and demonstrated promising antitumor efficacy, especially in patients with RAS/RAF/MEK/ERK pathway mutations. These findings support future clinical development of MEK inhibition strategies for BTC management.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed:

1. A method for treating biliary tract cancer comprising administering to a patient in need thereof, over a period of time, therapeutic agents that comprises an amount of a fluoropyrimidine-containing therapy and an amount of a MEK inhibitor or a pharmaceutically acceptable salt thereof, wherein the amounts together are effective in treating biliary tract cancer.

2. The method of claim 1, wherein the MEK inhibitor is binimetinib.

3. The method according to any one of claims 1-2, wherein the MEK inhibitor is crystallized binimetinib.

4. The method according to any one of claims 2-3, wherein binimetinib is administered orally in the amount of about 30 mg BID or about 45 mg BID during the period of time.

5. The method according to any one of claims 2-4, wherein binimetinib is administered orally in the amount of about 30 mg BID or about 45 mg BID for two weeks on and one week off in at least one treatment cycle of three weeks during the period of time.

6. The method according to any one of claims 2-5, wherein binimetinib is administered orally in the amount of about 30 mg BID.

7. The method according to any one of claims 1-5, wherein the fluoropyrimidine-containing therapy is a 5-FU prodrug.

8. The method according to claim 7, wherein the fluoropyrimidine-containing therapy is capecitabine.

9. The method according to claim 8, wherein capecitabine is administered orally in the amount of about 800 mg/m², 825 mg/m², or about 950 mg/m², or about 1000 mg/m², or about 1250 mg/m² twice daily.

10. The method according to claim 8 or 9, wherein capecitabine is administered orally in the amount of about 800 mg/m², 825 mg/m², or about 950 mg/m², or about 1000 mg/m², or about 1250 mg/m² twice daily for two weeks on and one week off in at least one treatment cycle of three weeks during the period of time.

11. The method according to claim 10, wherein capecitabine is administered orally in the amount of about 1250 mg/m² twice daily.

12. The method according to any one of claims 1-11, wherein, prior to the period of time, the patient was treated with a prior therapy

13. The method according to claim 12, wherein said prior therapy is selected from administration of one or more therapeutic agents independently selected from chemotherapeutic agents and targeted therapeutic agents.

14. The method according to claim 13, wherein the prior therapy was administration of gemcitabine as monotherapy.

15. The method according to claim 13, wherein the prior therapy was administration of gemcitabine and cisplatin.

16. The method according to claim 13, wherein the prior therapy was administration of gemcitabine and oxaliplatin.

17. The method according to claim 13, wherein the prior therapy was administration of 5-fluorouracil as a monotherapy.

18. The method according to claim 13, wherein the prior therapy was administration of 5-fluorouracil and leucovorin.

19. The method according to claim 13, the prior therapy was administration of 5-fluorouracil and cisplatin.

20. The method according to claim 13, wherein the prior therapy was administration of 5-fluorouracil, epirubicin, and cisplatin.

21. The method according to claim 13, wherein the prior therapy was administration of 5-fluorouracil and irinotecan.

22. The method according to claim 13, wherein the prior therapy was administration of capecitabine as a monotherapy.

23. The method according to claim 13, wherein the prior therapy was administration of gemcitabine.

24. The method according to claim 13, wherein the prior therapy was administration of gemcitabine and oxaliplatin.

25. The method according to claim 13, wherein the prior therapy was administration of lapatinib.

26. The method according to claim 13, wherein the prior therapy was administration of erlotinib.

27. The method according to claim 13, wherein the prior therapy was administration of cetuximab or panitumumab.

28. The method according to claim 13, wherein the prior therapy was administration of bevacizumab.

29. The method according to claim 13, wherein the prior therapy was administration of bevacizumab and mFOLFOX6.

30. The method according to claim 13, wherein the prior therapy was administration of bevacizumab in combination with gemcitabine and capecitabine.

31. The method according to claim 13, wherein the prior therapy was administration of a MEK inhibitor as a monotherapy.

32. The method according to claim 31, wherein the prior therapy was administration of selumetinib.

33. The method according to claim 31, wherein the prior therapy was administration of trametinib.

34. The method according to any one of claims 12-33, wherein the prior therapy was determined to be ineffective.

35. The method according to any one of claims 12-33, wherein the patient was determined to be resistant to the prior therapy.

36. The method according to any one of claims 1-35, wherein the method further comprises assessing efficacy of treatment during the period of time by determining one or more of inhibition of disease progression, inhibition of tumor growth, reduction of primary tumor, relief of tumor-related symptoms, inhibition of tumor secreted factors, delayed appearance of primary or secondary tumors, slowed development of primary or secondary tumors, decreased occurrence of primary or secondary tumors, slowed or decreased severity of secondary effects of disease, arrested tumor growth and regression of tumors, increased Time To Progression (TTP), increased Progression Free Survival (PFS), increased Overall Survival (OS) or increased Duration of Response (DOR).

37. The method according to any one of claims 1-36, wherein the biliary tract cancer has a KRAS mutation.

38. The method according to claim 37, wherein the biliary tract cancer has a KRAS G12A mutation.

39. The method according to claim 37, wherein the biliary tract cancer has a KRAS G12C mutation.

40. The method according to claim 37, wherein the biliary tract cancer has a KRAS G12D mutation.

41. The method according to claim 37, wherein the biliary tract cancer has a KRAS G12V mutation.

42. The method according to any one of claims 1-36, wherein the biliary tract cancer has a NRAS mutation.

43. The method according to claim 42, wherein the biliary tract cancer has a NRAS Q61L mutation.

44. The method according to any one of claims 1-36, wherein the biliary tract cancer has a MAP2K1 mutation.

45. The method according to claim 44, wherein the biliary tract cancer has a MAP2K1 E203K mutation.

46. The method according to claim 44, wherein the biliary tract cancer has a MAP2K1 E203V mutation.

47. The method according to any one of claims 1-46, wherein the biliary tract cancer is selected from intrahepatic cholangiocarcinoma and extrahepatic cholangiocarcinoma, and ampulla of Vater cancer.

48. The method according to any one of claims 1-47, wherein the biliary tract cancer is unresectable.

49. The method according to any one of claims 1-47, wherein the biliary tract cancer is recurrent.

50. A combination therapy method comprising administering, over a period of time, to a patient having biliary tract cancer, therapeutic agents that comprise therapeutically effective amounts, independently or in combination, of:
a MEK inhibitor or a pharmaceutically acceptable salt thereof; and
a fluoropyrimidine-containing therapy.

51. The combination therapy method according to claim 50, wherein the MEK inhibitor is binimetinib.

52. The method according to any one of claims 50-51, wherein the MEK inhibitor is crystallized binimetinib.

53. The combination therapy method according to any one of claims 50-52 wherein the fluoropyrimidine-containing therapy is capecitabine.

54. The combination therapy method according to any one of claims 51-53, wherein binimetinib is orally administered to the patient as a tablet during the period of time.

55. The combination therapy method according to claim 54, wherein said tablet comprises 15 mg of binimetinib.

56. The combination therapy according to any one of claims 53-55, wherein capecitabine is administered orally during the period of time.

57. The combination therapy according to claim 56, wherein capecitabine is administered as a tablet.

58. The combination therapy according to claim 57, where said tablet comprises 150 mg of capecitabine.

59. The combination therapy according to claim 57, where said tablet comprises 300 mg of capecitabine.

60. The combination therapy according to any one of claims 50-59, wherein the biliary tract cancer has a KRAS mutation.

61. The combination therapy according to claim 60, wherein the biliary tract cancer has a KRAS G12A mutation.

62. The combination therapy according to claim 60, wherein the biliary tract cancer has a KRAS G12C mutation.

63. The combination therapy according to claim 60, wherein the biliary tract cancer has a KRAS G12D mutation.

64. The combination therapy according to claim 60, wherein the biliary tract cancer has a KRAS G12V mutation.

65. The combination therapy according to any one of claims 50-59, wherein the biliary tract cancer has a NRAS mutation.

66. The combination therapy according to claim 65, wherein the biliary tract cancer has a NRAS Q61L mutation.

67. The combination therapy according to any one of claims 50-59, wherein the biliary tract cancer has a MAP2K1 mutation.

68. The combination therapy according to claim 67, wherein the biliary tract cancer has a MAP2K1 E203K mutation.

69. The combination therapy according to claim 67, wherein the biliary tract cancer has a MAP2K1 E203V mutation.

70. The method according to any one of claims 50-69, wherein the biliary tract cancer is selected from intrahepatic cholangiocarcinoma and extrahepatic cholangiocarcinoma, and ampulla of Vater cancer.

71. The method according to any one of claims 50-70, wherein the biliary tract cancer is unresectable.

72. The method according to any one of claims 50-70, wherein the biliary tract cancer is recurrent.
